(19) United States
(12) Patent Application Publication
(10) Pub. No.: US 2025/0327816 A1
(43) Pub. Date: Oct. 23, 2025

(54) BIOMARKERS FOR THE PREDICTION OF PRETERM BIRTH

(71) Applicants: UNIVERSITÉ LAVAL, Québec (CA); CEZANNE S.A.S., Nimes Cédex 1 (FR); B.R.A.H.M.S GMBH, Hennigsdorf (DE)

(72) Inventors: Emmanuel BUJOLD, Québec (CA); Bénedicte JARDIN-WATELET, Nimes Cédex 1 (FR); Delphine ESPINASSE, Nimes Cédex 1 (FR); Anne INCAMPS, Nimes Cédex 1 (FR); Michael HAUSMANN, Hennigsdorf (DE); Pauline REBILLARD, Nimes Cédex 1 (FR)

(73) Assignees: UNIVERSITÉ LAVAL, Québec (CA); CEZANNE S.A.S., Nimes Cédex 1 (FR); B.R.A.H.M.S GMBH, Hennigsdorf (DE)

(21) Appl. No.: 18/718,852

(22) PCT Filed: Dec. 13, 2022
(86) PCT No.: PCT/EP2022/085557

§ 371 Date: Jun. 12, 2024

(30) Foreign Application Priority Data
Dec. 13, 2021 (EP) 21214104.8

(51) Int. Cl. G01N 33/68

(52) U.S. Cl. CPC G01N 33/689; C12Y 304/24035; G01N 2333/96494; G01N 2800/368

(57) ABSTRACT

The present invention relates to clinical diagnostics including diagnosis, prognosis, prediction, risk assessment and/or risk stratification of preterm birth (PTB) and subsequent treatment in a pregnant subject, and corresponding methods and products. The invention provides decision tools to help clinicians choosing the most appropriate management for the pregnant women. In particular, the present invention relates to a method for the diagnosis, prognosis, prediction, risk assessment and/or risk stratification of preterm birth (PTB) in a pregnant subject, the method comprising determining a level of one or more biomarkers in a sample that has been isolated from said pregnant subject, wherein the one or more biomarkers comprise at least one of matrix metallopeptidase 9 (MMP9) or fragment(s) thereof and Pappalysin-2 (PAPP-A2) or fragment(s) thereof, wherein the level of the one or more biomarkers in the sample is indicative of the presence or absence of a subsequent PTB.

The present invention is in the field of clinical diagnostics. In particular, the invention relates to the diagnosis, prognosis, prediction, risk assessment and/or risk stratification of preterm birth (PTB) in a pregnant subject, and corresponding methods and products. The invention provides decision tools to help clinicians choosing the most appropriate management for the pregnant women. In particular, the present invention relates to a method for the diagnosis, prognosis, prediction, risk assessment and/or risk stratification of preterm birth (PTB) in a pregnant subject, the method comprising determining a level of one or more biomarkers in a sample that has been isolated from said pregnant subject, wherein the one or more biomarkers comprise at least one of matrix metallopeptidase 9 (MMP9) or fragment(s) thereof and Pappalysin-2 (PAPP-A2) or fragment(s) thereof, wherein the level of the one or more biomarkers in the sample is indicative of the presence or absence of a subsequent PTB.

### BACKGROUND OF THE INVENTION

The World Health Organisation (WHO) defines preterm birth (PTB) in human as any birth before 37 completed weeks of gestation. PTB is associated with significant morbidity and mortality. Premature babies born before 37 weeks, if they survive, have a high incidence of visual and hearing defects, lung disease, developmental delays, and cerebral palsy (Blencowe et al., 2013). The rate of preterm birth is estimated that over 15 million annually (Quinn et al., 2016).

Preterm birth is associated with significant costs to health systems, and families of preterm newborns often experience considerable psychological and financial hardship (Korvenranta et al., 2010). Although the risks of mortality and morbidity are much higher in early gestation (<34 weeks), late preterm birth (37<weeks) occurs more often, and newborn babies born late preterm have significantly higher risks of adverse outcomes than babies born at term (Chawanpaiboon et al., 2018). Preterm births are categorized as spontaneous preterm birth (sPTB) due to spontaneous preterm labour or preterm premature rupture of membranes (PPROM) (about 75% of all premature births) or as indicated preterm birth (iPTB) (about 25% of all premature births) occur as a result of maternal or fetal complications such as preeclampsia or gestational diabetes. Although many sociodemographic, nutritional, biological, and environmental factors can increase the risk of spontaneous preterm birth, the cause is not fully understood (Goldenberg et al., 2008).

Relatively little progress has been made in determining whether a pregnant women is at risk for sPTB. The lack of tools for identifying a risk early in pregnancy coupled with an inability to pinpoint underlying etiology prevents clinicians from proactively detecting and managing the at-risk pregnancy hampering their efforts to improve pregnancy outcomes (Quinn et al., 2016). There is a significant need to identify pregnant women who are at risk of sPTB.

In clinical practice, the high-risk pregnancies could be identified in the 1^{st} trimester (11-13 weeks of gestation) based on maternal characteristics and obstetric history (prior medical history or clinical examinations). This screening could detect <30% of preterm deliveries in women with previous pregnancies (multiparous) and <20% in those without previous pregnancy (nulliparous) at a false positive rate (FPR) of 10% (Beta et al., 2011). Thus, this screening allows to identify only a small subsection of the true high-risk pregnancies prone to preterm birth, and thereby leaves out the important group of women having their first pregnancy.

Currently the gold standard to identify women at risk of preterm birth is a mid-trimester (16-24 weeks of gestation) cervical length (CL) assessment by transvaginal ultrasound (see for example https://fetalmedicine.org/fmf-certification-2/cervical-assessment-1). The threshold used to determine a “short” cervix ranges from 15 to 30 mm depending on the population studied and the gestational age of assessment. The specificity of a short cervical length to predict preterm birth is related to the used cut-off. The detection rate (DR) in asymptomatic women without prior history of preterm birth ranges from 6% to 58% at a FPR of 1-3% (Son et al., 2017). Yet its clinical utility as a universal screening tool is currently the focus of much debate in obstetrics. Concerns over the quality and consistency of this measurement have limited the clinical use (Parry et al., 2012).

Additionally, different commercial tests are available, such as fetal fibronectin (fFN), PAMG-1 or IGFBP1. The principle of these tests is based on the collection of vaginal fluid with a swab to determine whether or not these molecules are present. These types of tests are used in symptomatic women population and are more relevant for a rule out approach as their negative predictive values are very high. However, the vaginal fluid collection can be an issue in addition to the fact that this approach is difficult to standardize. A rapid MMP8 point-of-care test is also available to identify intra-amniotic inflammation/infection and impending preterm delivery in patients with preterm labor and intact membranes.

Additionally, US 2008/02554490 A1 discloses assay methods for identifying an increased risk of spontaneous preterm birth (sPTB), which is specifically induced by PPROM. The assay is based on the determination of protease levels (i.e. metalloproteases such as MMP9) in saliva samples. Lee et al., 2015 discloses further a combination of MMP-9 and inflammatory markers such as IL-8 as biomarkers for the risk of preterm birth in women carrying twins, wherein biomarker levels are determined in amniotic fluid.

CA 2990000 A1 discloses the determination of biomarker combinations of PSG3 and other proteins for determining the risk of preterm birth. Parry et al., 2019 further disclose the determination of PAPP-A1 and PAPP-A2 in serum and placenta samples collected from women who had spontaneous, drug-induced preterm birth or a term birth.

Methods are available for the diagnosis of preeclampsia as one of multiple possible maternal complications that can lead to preterm birth (iPTB). Such a method is disclosed in US 2010/016173 A1, which teaches MMP-9 and PAPP-A2 as biomarkers in serum for preeclampsia. US 214/141456 A1 teaches that PAPP-A2 in combination with further biomarkers such as PAPP-A are determined in blood, plasma, serum and saliva samples. Further biomarkers for preeclampsia are disclosed by Rasanen et. Al, 2010, disclosing a combination of PAPP-A2, fibronectin, and MMP-9 determined in serum. The association of MMP-9 and PAPP-A2 with the development of preeclampsia is further disclosed by Shah et al., 2015.

Several strategies can be employed to prevent/delay preterm birth or mitigate the consequences such as bed rest, close monitoring, vaginal progesterone, cervical cerclage, tocolysis, antibiotics or corticosteroids administration to promote the fetal lung development. However, the effectiveness of these strategies depends on the ability to accurately predict as early as possible and in all pregnant women (multiparous and nulliparous, asymptomatic and symptomatic) which women is at increased risk of preterm delivery.

Accordingly, there is a need in the art to provide improved means for identifying subjects that are at risk of PTB, preferably across all pregnancy trimesters, especially in asymptomatic subjects without PTB history. Ideally, methods and products should be developed that can be used in practically all pregnant women (nulliparous and multiparous, asymptomatic and symptomatic) and across all trimesters of pregnancy, especially early in pregnancy (1^{st} trimester), which are based on easily accessible and standardized biological sample (blood vs vaginal fluid, amniotic fluid or saliva for other biomarkers), which does not require highly trained healthcare staff as it's required for CL assessment, and which are associated with higher detection rates than current clinical practice, especially cervical length assessment Until now, no reliable methods that fullfil the above requirements are available.

### SUMMARY OF THE INVENTION

In light of the limitations of the methods and products of the prior art, the technical problem underlying the present invention is the provision of means for diagnosis, prognosis, prediction, risk assessment and/or risk stratification of preterm birth (PTB) in a pregnant subject.

The present invention therefore relates to methods, kits and further means for diagnosis, prognosis, prediction, risk assessment and/or risk stratification of preterm birth (PTB) in a pregnant subject. The method may also be used as a method of therapy guidance, therapy stratification and/or therapy control in a pregnant subject identified to have an increased risk of PTB, which has preferably been identified by using the means of the present invention.

One object of the invention is therefore the use of a biomarker, or combination of biomarkers to distinguish patients who are more likely or have a high risk of PTB and that may require preventive, symptomatic or causative treatment, from subjects who have a low risk of PTB and not requiring such treatment.

The solution to the technical problem of the invention is provided in the independent claims. Preferred embodiments of the invention are provided in the dependent claims.

In one aspect the invention relates to a method for the diagnosis, prognosis, prediction, risk assessment and/or risk stratification of preterm birth (PTB) in a pregnant subject, the method comprising:
determining a level of one or more biomarkers in a sample that has been isolated from said pregnant subject,
wherein the one or more biomarkers comprise at least one of matrix metallopeptidase 9 (MMP9) or fragment(s) thereof and Pappalysin-2 (PAPP-A2) or fragment(s) thereof,
wherein the level of the one or more biomarkers in the sample is indicative of the presence or absence of a subsequent PTB.

In one embodiment the invention relates to a method for the diagnosis, prognosis, prediction, risk assessment and/or risk stratification of preterm birth (PTB) in a pregnant subject, the method comprising:
determining a level of matrix metallopeptidase 9 (MMP9) in a sample that has been isolated from said pregnant subject,
wherein the level of MMP9 in the sample is indicative of the presence or absence of a subsequent PTB.

In one embodiment the invention relates to a method for the diagnosis, prognosis, prediction, risk assessment and/or risk stratification of preterm birth (PTB) in a pregnant subject, the method comprising:
determining a level of Pappalysin-2 (PAPP-A2) in a sample that has been isolated from said pregnant subject,
wherein the level of PAPP-A2 in the sample is indicative of the presence or absence of a subsequent PTB.

The present invention is based on the entirely surprising finding that PAPP-A2 and/or MMP9 are predictive biomarkers for spontaneous preterm birth throughout pregnancy. Surprisingly, PAPP-A2 and/or MPP9, as single biomarkers, in combination with each other, or in combination with another biomarker, are prognostic biomarkers.

As shown herein, PAPP-A2 and MMP9 allow, as single biomarkers, or in combination with each other, or in combination with another biomarker, the detection and prediction of all preterm birth, in particular spontaneous preterm birth, from first through third trimester. In particular, PAPP-A2 and/or MMP9 allow, as single biomarker, or in combination with each other, or in combination with another biomarker, the identification of preterm birth in the first trimester, but also during the second or third trimester, and the markers are particularly useful for detecting early preterm birth (<34 weeks) and very early preterm birth (<32 weeks). In embodiments, the level of PAPP-A2 and/or MMP9 can be detected in a blood sample, such as preferably a serum or plasma sample.

In embodiments, the level of MMP9 can be detected in a blood sample, such as preferably a serum or plasma sample.

In embodiments, the level of PAPP-A2 can be detected in a blood sample, such as preferably a serum or plasma sample.

In contrast, methods known in the prior art are performed using vaginal fluid, amniotic fluid or saliva samples, which are not standardized with respect to the sample size taken, whereby in particular collecting too little sample can lead to false results. Further, these methods require highly trained healthcare stuff for sample acquisition and processing, as e.g., the acquisition of amniotic fluid and vagial fluid can bear risks for pregnant women. It is a great advantage of the method of the invention that it provides a biomarker based PTB predictive and prognostic test that can be performed using standard blood based samples that can be obtained easily in a standardized way.

In embodiments, the level of the one or more biomarkers in said sample is compared to a reference level of said biomarker, wherein preferably the reference level is derived from pregnant subjects without a prenatal disorder or condition, preferably without PTB.

Accordingly, in embodiments the determined levels of the biomarkers of the present invention are compared to the levels of biomarkers that have been determined in healthy controls (reference group), which preferably are in the context of the present invention pregnant subjects without PTB or preferably without any prenatal disorder or prenatal condition or pregnancy complication. In embodiments, the control group is composed of pregnant subjects that are known to have not experienced any pregnancy complications, or subjects that are known to have not experienced PTB. In embodiments, the reference group may be subdivided into age groups, and/or may be subdivided into groups considering the number of pregnancies. In embodiments, the reference level and/or reference data comprise or corresponds to a level of the respective biomarker as determined in the control/reference group at the respective time point of gestation, for example a mean or median.

In preferred embodiments, the determining of a biomarker in the sample from the pregnant subject of the method of the invention is performed by the same method and using the same equipment that has been used for determining the reference levels in the control group of subjects without prenatal disorder or condition or PTB.

In embodiments, the comparability of the determined biomarker level to a reference level is ensured. It is not absolutely necessary that the same equipment or method of determining the biomarker level in a sample is used as long as suitable adjustments or comparison methods are established to ensure comparability of the determined level to a reference level.

In embodiments, using reference data of the invention can comprise a threshold level or threshold value which can be calculated by comparing determined biomarker levels in a control group versus determined biomarker levels in a group of pregnant subjects that experience PTB. Such a threshold, also termed reference level, can be an absolute value, such as a population mean and/or a population median of a biomarker level, or can be a fold change of multiple of mean and/or median over the mean and/or median of the control group. In embodiments, the reference level is a population mean and/or a population median of MMP9 and/or PAPP-A2 levels, or is calculated from a population mean and/or a population median of MMP9 and/or PAPP-A2 levels.

In embodiments the reference level is a population mean and/or a population median of MMP9 levels, or is calculated from a population mean and/or a population median of MMP9 levels.

In embodiments the reference level is a population mean and/or a population median of PAPP-A2 levels or is calculated from a population mean and/or a population median of PAPP-A2 levels.

In embodiments, the reference level can be one or more median values from table 4 of the examples below, and/or can be calculated from the median values of table 4. In embodiments, the reference level can be the median values from a normal pregnancy at any given time point of table 4, and/or can be calculated from the median values from a normal pregnancy, at any given time point of table 4. In embodiments, the reference level can be one or more median values from table 4 of the examples below, with a ±85%, ±80%, ±70%, ±60, ±50%, ±40%, ±30%±20%, ±10%, or ±5% variation from the specific value listec in the table.

In embodiments, an increased level of the one or more biomarkers in the pregnant subject as compared to the reference level is indicative of a subsequent PTB. Such embodiments are advantageous, as they allow the identification of pregnant subjects that are at risk of PTB later during their pregnancy and it is possible to take preventive measures for these subjects to reduce the risk or prevent PTB later during pregnancy. Accordingly, the method of the invention can be used to assign an increased risk of future PTB to a subject that would otherwise be difficult to identify by using previous method of PTB prediction of the state of the art.

In embodiments, the method of the invention may include treatment guidance and can enable improved management for a subject that has been identified to have an increased risk of PTB by using the method of the invention. In such embodiments, the method of the invention can be used to prevent or at least reduce the risk of PTB and preventive medical support can be provided, for example to improve development of the fetus and to preserve the pregnancy as long as possible.

In embodiments, a level of the one or more biomarkers in the pregnant subject that is not increased as compared to the reference levels is indicative of the absence of a subsequent PTB. In embodiments, an unchanged or even decreased level of the one or more biomarkers in the pregnant subject as compared to the reference level is indicative of the absence of a subsequent PTB. Such embodiments of the invention are particularly useful to identify subjects that may not be at an increased risk of PTB. It is advantageous to have such a possibility of ruling out an increased risk of PTB as the available resources for pregnancy supervision can be allocated to those subjects that may have an increased risk, while those which have no increased risk as compared to the health control group do not have undergo further additional or non-standard test during pregnancy. In embodiments, subjects that according to the method of the present invention have no increased risk of PTB only should to attend regular pregnancy screenings and check points but may not require additional testing or PTB preventive measures.

In embodiments, the reference level may be the median level of the respective biomarker as determined in the control group, and the comparison of the determind level to the reference levels in such embodiments can be the determining of the multiples of the median (MoM) of the determined biomarker level as compared to the median of the control group. In embodiments the median levels disclosed in table 4 of the present invention may be a reference level according to the present invention. In further embodiments the median values from a normal pregnancy at any given time point of table 4 may be a reference level according to the present invention. In embodiment, a significant increase of the level or a higher level of the biomarker in the sample from the subject of the method of the invention in comparison to the median of the biomarker in the control group can be indicative of the presence of a subsequent PTB. In embodiments, absence of an increase or a higher level in comparison to the median of the control group can be indicative of the absence of a subsequent PTB.

In embodiments the reference level is a is a population median of MMP9 and/or PAPP-A2 levels from a normal pregnancy determined in a blood sample, preferably a serum or plasma sample. In embodiments the reference level is a is a population median of MMP9 and/or PAPP-A2 levels from a normal pregnancy determined in a blood sample, preferably a serum or plasma sample isolated in gestational weeks 11 to 13. In embodiments the reference level is a is a population median of MMP9 and/or PAPP-A2 levels from a normal pregnancy determined in a blood sample, preferably a serum or plasma sample isolated in gestational weeks 20 to 24. In embodiments the reference level is a is a population median of MMP9 and/or PAPP-A2 levels from a normal pregnancy determined in a blood sample, preferably a serum or plasma sample isolated in gestational weeks 30 to 34.

In embodiments the reference level is a population median of MMP9 levels from a normal pregnancy determined as MMP9 abundance level in a blood sample isolated in gestational week 11 to 13. In embodiments the reference level determined as abundance level of MMP9 in a blood sample isolated in gestational week 11 to 13 is 13.4±50%, preferably 13.4±46.3%. In embodiments the reference level determined as abundance level of MMP9 in a blood sample isolated in gestational week 11 to 13 is 13.4±50%, 13.4±40%, 13.4±30%, 13.4±20%, 13.4±15%, 13.4±10%, 13.4±5% or 13.4±2.5%.

In embodiments the reference level is a population median of MMP9 levels from a normal pregnancy determined as MMP9 abundance level in a blood sample isolated in gestational week 20 to 24. In embodiments the reference level determined as abundance level of MMP9 in a blood sample isolated in gestational week 20 to 24 is 16.0±40%, preferably 16.0±38.1%. In embodiments the reference level determined as abundance level of MMP9 in a blood sample isolated in gestational week 20 to 24 is 16.0±40%, 16.0±30%, 16.0±20%, 16.0±15%, 16.0±10%, 16.0±5% or 16.0±2.5%.

In embodiments the reference level is a population median of MMP9 levels from a normal pregnancy determined as MMP9 abundance level in a blood sample isolated in gestational week 30 to 34. In embodiments the reference level determined as abundance level of MMP9 in a blood sample isolated in gestational week 30 to 34 is 15.7±40%, preferably 15.7±36.9%. In embodiments the reference level determined as abundance level of MMP9 in a blood sample isolated in gestational week 30 to 34 is 15.7±40%, 15.7±30%, 15.7±20%, 15.7±15%, 15.7±10%, 15.7±5% or 15.7±2.5%.

In embodiments the reference level is a population median of PAPP-A2 levels from a normal pregnancy determined as PAPP-A2 concentration in a blood sample isolated in gestational week 11 to 13. In embodiments the reference level determined as PAPP-A2 concentration in a blood sample isolated in gestational week 11 to 13 is 14.1±50% ng/ml, preferably 14.1±49.6% ng/ml. In embodiments the reference level determined as PAPP-A2 concentration in a blood sample isolated in gestational week 11 to 13 is 14.1±50%, 14.1±40%, 14.1±30%, 14.1±20% ng/ml, 14.1±15% ng/ml, 14.1±10% ng/ml, 14.1±5% ng/ml or 14.1±2.5% ng/ml.

In embodiments the reference level is a population median of PAPP-A2 levels from a normal pregnancy determined as PAPP-A2 concentration in a blood sample isolated in gestational week 20 to 24. In embodiments the reference level determined as PAPP-A2 concentration in a blood sample isolated in gestational week 20 to 24 is 17.5±85% ng/ml, preferably 17.5±84% ng/ml. In embodiments the reference level determined as PAPP-A2 concentration in a blood sample isolated in gestational week 20 to 24 is 17.5±85%, 17.5±80%, 17.5±70%, 17.5±60%, 17.5±50%, 17.5±40%, 17.5±30%, 17.5±20% ng/ml, 17.5±15% ng/ml, 17.5±10% ng/ml, 17.5±5% ng/ml or 17.5±2.5% ng/mL.

In embodiments the reference level is a population median of PAPP-A2 levels from a normal pregnancy determined as PAPP-A2 concentration in a blood sample isolated in gestational week 30 to 34. In embodiments the reference level determined as PAPP-A2 concentration in a blood sample isolated in gestational week 30 to 34 is 44.9±90% ng/ml, preferably 44.9±86.4% ng/ml. In embodiments the reference level determined as PAPP-A2 concentration in a blood sample isolated in gestational week 30 to 34 is 44.9±90%, 44.9±80%, 44.9±70%, 44.9±60%, 44.9±50%, 44.9±40%, 44.9±30%, 20% ng/ml, 44.9±15% ng/ml, 44.9±10% ng/ml, 44.9±5% ng/ml or 44.9±2.5% ng/mL.

In the context of the present invention any numeric value such as a reference level may in embodiments comprise the numeric value±2.5%, 5%, 10%, 15%, 20%, 30%, 40%, 50%, 60%, 70%, 80%, 85% or 90%.

In embodiments, a determined level of the biomarkers, i.e. MMP9 or PAPP-A2, that is 10%, 20, 30, 40, 50, 60, 70, 80, 90, 100, 110, 120, 130, 140, 150, 160, 180, 200, 220, 240, 260, 280, 300, 320, 340, 360, 380, 400, 420, 440, 460, 480, 500% higher than the mean or median of the control population is indicative of the presence of a subsequent PTB. It is understood that a level that is 100% than the median is 2-fold the median, whereas a 10% increase is 1.1-fold the median.

In embodiments, a determined level of MMP9, that is 10%, 20, 30, 40, 50, 60, 70, 80, 90, 100, 110, 120, 130, 140, 150, 160, 180, 200, 220, 240, 260, 280, 300, 320, 340, 360, 380, 400, 420, 440, 460, 480, 500% higher than the mean or median of the control population is indicative of the presence of a subsequent PTB. It is understood that a level that is 100% than the median is 2-fold the median, whereas a 10% increase is 1.1-fold the median.

In embodiments, a determined level of PAPP-A2, that is 10%, 20, 30, 40, 50, 60, 70, 80, 90, 100, 110, 120, 130, 140, 150, 160, 180, 200, 220, 240, 260, 280, 300, 320, 340, 360, 380, 400, 420, 440, 460, 480, 500% higher than the mean or median of the control population is indicative of the presence of a subsequent PTB. It is understood that a level that is 100% than the median is 2-fold the median, whereas a 10% increase is 1.1-fold the median.

In embodiments, the reference level may be the mean level of the respective biomarker as determined in the control group, and the comparison of the determined level to the reference levels can in such embodiments be the determining of the fold change (FC) of the determined biomarker level as compared to the mean or median of the control group. In embodiments the FC values disclosed in table 4 of the present invention may be a reference level according to the present invention. In embodiment, an increase/higher of the level of the biomarker in the sample from the subject of the method of the invention in comparison to the mean or median of the biomarker in the control group can be indicative of the presence of a subsequent PTB. In embodiments, absence of a significant increase/higher level in comparison to the mean or median of the control group can be indicative of the absence of a subsequent PTB.

In one embodiment a determined level of MMP9, that is 30% higher (FC 1.3), in a blood sample isolated in gestational week 11 to 13 than the median of the control population is indicative of the presence of a subsequent PTB in all sPTB. In one embodiment, a determined level of MMP9, that is 50% higher (FC 1.5), in a blood sample isolated in gestational week 11 to 13 than the median of the control population is indicative of the presence of a subsequent PTB before 32 weeks of gestation (early PTB) or before 34 weeks of gestation (very early PTB

In one embodiment, a determined level of MMP9, that is 20% higher (FC 1.2), in a blood sample isolated in gestational week 20 to 24 than the median of the control population is indicative of the presence of a subsequent PTB in all sPTB. In one further embodiment, a determined level of MMP9, that is 60% higher (FC 1.6), in a blood sample isolated in gestational week 20 to 24 than the median of the control population is indicative of the presence of a subsequent PTB before 34 weeks of gestation.

In one embodiment, a determined level of MMP9, that is 20% higher (FC 1.2), in a blood sample isolated in gestational week 30 to 34 than the median of the control population is indicative of the presence of a subsequent PTB in all sPTB. In one further embodiment, a determined level of MMP9, that is 40% higher (FC 1.4), in a blood sample isolated in gestational week 30 to 34 than the median of the control population is indicative of the presence of a subsequent PTB before 34 weeks of gestation.

In one embodiment, a determined level of PAPP-A2, that is 30% higher (FC 1.3), in a blood sample isolated in gestational week 20 to 24 than the median of the control population is indicative of the presence of a subsequent PTB in all sPTB. In one further embodiment, a determined level of PAPP-A2, that is 100% higher (FC 2.0), in a blood sample isolated in gestational week 20 to 24 than the median of the control population is indicative of the presence of a subsequent PTB before 34 weeks of gestation.

In one embodiment, a determined level of PAPP-A2, that is 120% higher (FC 2.2), in a blood sample isolated in gestational week 30 to 34 than the median of the control population is indicative of the presence of a subsequent PTB in all sPTB. In one further embodiment, a determined level of PAPP-A2, that is 260% higher (FC 3.6), in a blood sample isolated in gestational week 30 to 34 than the median of the control population is indicative of the presence of a subsequent PTB before 34 weeks of gestation.

In embodiments using MoM comparison or FC comparison, there can be specific cut-off levels (for example in percent or fold increase over the median or mean), above which a determined level is indicative of a subsequent PTB, wheres levels that are equal or below such a cut-off level can be indicative of the absence of a subsequent PTB.

In embodiments, it is possible to use predetermined cut-off value/level (herein also called threshold value/level) for a biomarker of the method of the invention as a reference level to which the determined biomarker level is compared. In such embodiment, a determined biomarker level above the predetermined cut-off value may be indicative of the presence of a subsequent PTB, whereas a value below the cut-off value may be indicative of the absence of a subsequent PTB. Threshold levels may be predetermined absolute concentrations of the respective biomarker in a sample, or may be relative threshold levels, such as FC or MoM thresholds.

In embodiments, a level of the one or more biomarkers, i.e. MMP9 and/or PAPP-A2, are determined at different time points of gestation of the same pregnant subject, and the level at a later time point can be compared to a level determined at an earlier time point.

In embodiments, a level of the one or more biomarkers, i.e. MMP9, are determined at different time points of gestation of the same pregnant subject, and the level at a later time point can be compared to a level determined at an earlier time point.

In embodiments, a level of the one or more biomarkers, i.e. PAPP-A2, are determined at different time points of gestation of the same pregnant subject, and the level at a later time point can be compared to a level determined at an earlier time point.

In such embodiments, it is possible to determine an increase or a decrease of the respective biomarker of a specific time course during gestation, and a diagnosis or risk assessment can be carried out based on the change of the biomarker level in said patient.

In embodiments a level of the one or more biomarkers, i.e. MMP9 or fragment(s) thereof and/or PAPP-A2 or fragment(s) thereof are determined and processed in a computer-implemented method such as an algorithm. In embodiments the computer-implemented method is configured for comparing the determined levels of one or more biomarkers to each other and/or with a reference level. In embodiments the determined levels of the one or more biomarkers, i.e. MMP9 or fragment(s) thereof and/or PAPP-A2 or fragment(s) thereof are compared to each other or with a reference level in a computer implemented method.

In embodiments the computer-implemented method provides an automated statement on an estimated risk of a subject for PTB at any given time point of pregnancy. In embodiments the automated statement on an estimated risk of PTB is provided by comparing a level of one or more biomarkers to each other and/or with a reference level. In embodiments MMP9 or fragment(s) thereof to a reference level. In embodiments the automated statement on an estimated risk of PTB is provided by comparing a level of PAPP-A2 or fragment(s) thereof to a reference level. In embodiments the automated statement on an estimated risk of PTB is provided by comparing a level of MMP9 or fragment(s) thereof and a level of PAPP-A2 or fragments thereof to each other and/or a reference level. Im embodiments the level of the one or more biomarkers i.e. MMP9 and/or PAPP-A2 are determined at the same and/or different time points, i.e., in a blood sample isolated in gestational week 11 to 13, gestational week 20 to 24 and/or gestational week 30 to 34. In embodiments the level of the one or more biomarkers i.e. MMP9 and/or PAPP-A2 are determined in multiple measurements at the same or different time point, i.e., in a blood sample isolated in gestational week 11 to 13, gestational week 20 to 24 and/or gestational week 30 to 34.

According to the present invention, the term “indicate” in the context of “indicative of the presence or absence of subsequent PTB” is intended as a measure of risk and/or likelihood. Preferably, the “indication” of the presence or absence of PTB is intended as a risk assessment, and is typically not to be construed in a limiting fashion as to point definitively to the absolute presence or absence of said event.

Therefore, the term “indicative of the absence of PTB” can be understood as indicating a low or high risk of the occurrence of PTB, respectively.

It was entirely surprising that a level of MMP9 and/or PAPP-A2 or fragments thereof could be correlated with the likelihood of the presence or absence of subsequent PTB.

In embodiment of the invention, PTB is spontaneous PTB (sPTB) or indicated preterm birth (iPTB), preferably sPTB.

In embodiments of the invention, PTB is early sPTB before gestational week 34 or very early sPTB before gestational week 32.

As used herein, PTB relates to PTB before week 37 and comprises early PTB before week 34 and very early PTB before week 32. As used herein, the term late PTB may relate to PTB before week 37 and during or after week 34 (34</=PTB<37).

In embodiments, the sample has been isolated from the pregnant subject in the first, second or third trimester of pregnancy, preferably in gestational weeks 9-13 or 11-13 in the first trimester, or 20-24 in the second trimester, or 30-34 in the third trimester, respectively.

In embodiments, a sample has been isolated from the pregnant subject in gestational weeks 9-14, 11-14, 9-13, or 11-13.

In embodiments, a sample has been isolated from the pregnant subject in gestational weeks 20-37, 20-34, 20-27, or 20-24.

In embodiments, a sample has been isoldated from the pregnant subject in gestational weeks 20-37, 30-37, or 30-24.

in the first, second or third trimester of pregnancy, preferably in gestational weeks 9-13 or 11-13 in the first trimester, or 20-24 in the second trimester, or 30-34 in the third trimester, respectively.

In embodiments, the sample has been isolated in gestational week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and/or 37.

It is a great advantage of the method of the invention that it is possible to provide a prediction and/or risk assessment of PTB during early periods of pregnancy, and in particular before term, i.e. before week 38. Accordingly, in embodiments of the invention the sample is isolated before week 38 of gestation. Methods of the state of the art can often only predict labor at term. However, the method of the invention provides means for predicting PTB based on samples that can be isolated during the first, second and/or third trimester of pregnancy and before term. The present invention is based on the surprising finding that determining the biomarkers of the invention, in particular MMP9 and/or PAPP-A2, in a sample of a pregnant subject enable the prediction of PTB, in particular sPTB several weeks and even months before the onset of PTB.

In preferred embodiments, the pregnant subject is a nulliparous woman. Such embodiments represent a particular further development since existing clinical screenings used for PTB prediction of the state of the art are in particularly inexact for nulliparous women with no previous pregnancies.

In embodiments, the pregnant subject is a multiparous woman.

In embodiments, the pregnant subject shows no signs of PTB (asymptomatic subject). In embodiments, the pregnant subject showed no signs of PTB the time point of sample isolation (meaning that the subject was asymptomatic with respect to PTB when the sample was isolated).

In embodiments, the subject shows signs of PTB (symptomatic subject). In embodiments, the pregnant subject showed signs of PTB the time point of sample isolation (meaning that the subject was symptomatic with respect to PTB when the sample was isolated).

In preferred embodiments, the subject has a singleton pregnancy.

In embodiments, the sample is a bodily fluid sample, such as a blood sample, such as a venous blood sample, a capillary blood sample, a serum sample or a plasma sample, a vaginal fluid sample, a saliva sample or an amniotic fluid sample, preferably a blood sample, more preferably a serum sample or a plasma sample.

In preferred embodiments, the sample is a blood sample, more preferably a serum or a plasma sample. It is a great advantage of the method of the present invention that it is possible to determine a risk of a subsequent PTB based on determining a biomarker in a blood sample, such as in a blood derived serum or plasma sample. Such samples are easily obtainable in a standardized way and are more reliable and associated with a lower risk than for example vaginal fluid swaps, amniotic fluid sample isolation or other invasive intrauterine procedures as known from methods of the prior art.

In embodiments of the invention, the method comprises determining a level of at least two biomarkers in a sample, wherein the at least two biomarkers comprise at least one of MMP9 and Pappalysin-2 (PAPP-A2) or fragment(s) thereof, and optionally one or more of LTF, CGA, INHA, ACTA, NOTUM, PAPP-A, PSG3, MST1L, SHBG, sFIt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1, MMP8, or fragment(s) thereof.

In embodiments of the invention, the method comprises determining a level of at least two biomarkers in a sample, wherein the at least two biomarkers comprise MMP9 or fragment(s) thereof, and one or more of PAPP-A2, LTF, CGA, INHA, ACTA, NOTUM, PAPP-A, PSG3, MST1L, SHBG, sFlt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1, MMP8, or fragment(s) thereof.

In embodiments of the invention, the method comprises determining a level of at least two biomarkers in a sample, wherein the at least two biomarkers comprise Pappalysin-2 (PAPP-A2) or fragment(s) thereof, and one or more of MMP9, LTF, CGA, INHA, ACTA, NOTUM, PAPP-A, PSG3, MST1L, SHBG, sFIt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1, MMP8, or fragment(s) thereof.

The additional determining of one of more of the following markers that have been implicated or associated or correlated with pregnancy complications is particularly advantageous for increasing the predictive value of the method of the present invention. Such markers include besides MMP9 and/or PAPP-A2 one or more of LTF, CGA, INHA, ACTA, NOTUM, PAPP-A, PSG3, MST1L, SHBG, sFlt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1, MMP8, or fragment(s) thereof.

In embodiments, the invention comprises determining a level of at least one biomarker in a sample, wherein the at least one biomarkers comprises at least one of MMP9 and Pappalysin-2 (PAPP-A2) or fragment(s) thereof, and optionally at least one further clinical parameter, such as clinical history, weight, body mass index (BMI), result of cervical examination (e.g. cervical length or cervical shape), result of ultra sound or other clinical imaging method, age of the pregnant subject, Fetal Medicine Foundation (FMF) algorithms (https://fetalmedicine.org/fmf-certification-2/cervical-assessment-1), smoking habits, blood pressure, etc.

In embodiments, the invention comprises determining a level of MMP9 or fragment(s) thereof, and optionally at least one further clinical parameter, such as clinical history, weight, body mass index (BMI), result of cervical examination (e.g. cervical length or cervical shape), result of ultra sound or other clinical imaging method, age of the pregnant subject, Fetal Medicine Foundation (FMF) algorithms (https://fetalmedicine.org/fmf-certification-2/cervical-assessment-1), smoking habits, blood pressure, etc.

In embodiments, the invention comprises determining a level of Pappalysin-2 (PAPP-A2) or fragment(s) thereof, and optionally at least one further clinical parameter, such as clinical history, weight, body mass index (BMI), result of cervical examination (e.g. cervical length or cervical shape), result of ultra sound or other clinical imaging method, age of the pregnant subject, Fetal Medicine Foundation (FMF) algorithms (https://fetalmedicine.org/fmf-certification-2/cervical-assessment-1), smoking habits, blood pressure, etc.

It was surprisingly found that determining at least one of the MMP9 and/or Pappalysin-2 (PAPP-A2) or fragment(s) thereof and at least one additional biomarker or clinical parameter leads to a unexpected improvement of the predictive value of the method of the invention. This synergistic effect of using more than one biomarker and/or clinical parameter was completely unforeseen. In particular, the synergistic effects observed in the examples as reported herein are surprising and were not obvious in view of the state of the art.

It is evident that the method of the present invention can be combined with other clinical parameters and known diagnostic tools that have been reported to be relevant or that are plausibly relevant for PTB as assessed by a skilled person to improved the predictive value for PTB. It is possible to combine the determined biomarkers of the invention with further biomarkers or clinical parameters in a diagnostic algorithm that can provide a more reliable risk assessment of the subject for PTB development.

In preferred embodiments, the invention comprises determining a level of at least two biomarkers in a sample, wherein the at least two biomarkers comprise at least one of MMP9 and Pappalysin-2 (PAPP-A2) or fragment(s) thereof, and optionally one or more of LTF, CGA, INHA, ACTA, NOTUM, PAPP-A, PSG3, MST1L, SHBG, sFlt-1, ADA12 and FCN3, or fragment(s) thereof.

In preferred embodiments, the invention comprises determining a level of at least two biomarkers in a sample, wherein the at least two biomarkers comprise MMP9 or fragment(s) thereof, and one or more of PAPP-A2, LTF, CGA, INHA, ACTA, NOTUM, PAPP-A, PSG3, MST1L, SHBG, sFlt-1, ADA12 and FCN3, or fragment(s) thereof.

In preferred embodiments, the invention comprises determining a level of at least two biomarkers in a sample, wherein the at least two biomarkers comprise Pappalysin-2 (PAPP-A2) or fragment(s) thereof, and one or more of MMP9, LTF, CGA, INHA, ACTA, NOTUM, PAPP-A, PSG3, MST1L, SHBG, sFlt-1, ADA12 and FCN3, or fragment(s) thereof.

As shown in the examples below, determining at least one of LTF, CGA, INHA, ACTA, NOTUM, PAPP-A, PSG3, MST1L, SHBG, sFIt-1, ADA12 and FCN3, or fragment(s) thereof in addition to MMP9 and/or PAPP-A2 increases the accuracy of the method of the invention.

Further, as shown in the examples below, determining at least one of PAPP-A2, LTF, CGA, INHA, ACTA, NOTUM, PAPP-A, PSG3, MST1L, SHBG, sFIt-1, ADA12 and FCN3, or fragment(s) thereof in addition to MMP9 increases the accuracy of the method of the invention. As shown in the examples below, determining at least one of MMP9, LTF, CGA, INHA, ACTA, NOTUM, PAPP-A, PSG3, MST1L, SHBG, sFIt-1, ADA12 and FCN3, or fragment(s) thereof in addition to PAPP-A2 increases the accuracy of the method of the invention.

In preferred embodiments, the one or more biomarkers comprise MMP9 or fragment(s) thereof.

In embodiments, the levels MMP9 or fragment(s) thereof is determined in a sample that has been isolated in the first, second or third trimester of pregnancy. In embodiments, the levels MMP9 or fragment(s) thereof is determined in a sample that has been isolated in the first, second or third trimester of pregnancy, wherein preferably no further biomarker is determined. In preferred embodiments, the levels MMP9 or fragment(s) thereof is determined in a sample that has been isolated in the first or second trimester of pregnancy, wherein preferably no further biomarker is determined.

It was completely unexpected that in embodiments it is possible to predict all kinds of spontaneous PTB within the 1^{st} trimester, preferably in weeks 9-13 of gestation, more preferably in weeks 11-13 of gestation, using MMP9 as a single biomarker or in combination with other biomarkers or clinical parameters. Furthermore, surprisingly MMP9 can also predict sPTB, in particular early PTB, in case of sample isolation in the second or third trimester, such as during weeks 20-24 or 30-34, respectively.

In embodiments, the method of the invention determines MMP9 or fragment(s) thereof as a single biomarker (meaning that no other biomarker is determined in the context of the method of the invention). In such embodiments, the sample may have been isolated during the first trimester, such as week 9-13 or preferably week 11-13 of gestation, and PTB may be before week 37 or early PTB or very early PTB, preferably early PTB and most preferably very early PTB.

In embodiments, the method of the invention determines MMP9 or fragment(s) thereof as a single biomarker and the sample has been isolated during the second trimester, preferably week 20-24 of gestation, and PTB may be before week 37 or early PTB, preferably early PTB.

In embodiments, the method of the invention determines MMP9 or fragment(s) thereof as a single biomarker and the sample has been isolated during the third trimester, preferably week 30-34 of gestation, and PTB may be before week 37 or early PTB, preferably early PTB.

In embodiments, the method of the invention determines MMP9 or fragment(s) thereof and at least one further biomarker, preferably selected from the list comprising of PAPP-A2, LTF, CGA, INHA, ACTA, NOTUM, PAPP-A, PSG3, MST1L, SHBG, sFIt-1, ADA12 and FCN3, PIGF, PCT, MR-proADM, MIP-1a, MIP-1b, estriol, TNF-a, IL-6, IL-8, IL-1b, AFP, fFN, PAMG1, phIGFBP1, MMP8, or fragment(s) thereof. It was found herein that combining determination of MMP9 with further biomarkers can increase the predictive value of the method.

In embodiments, the method of the invention determines MMP9 or fragment(s) thereof and at least one further biomarker, preferably selected from the list comprising PAPP-A2, LTF, CGA, INHA, ACTA, NOTUM, PAPP-A, PSG3, MST1L, SHBG, sFlt-1, ADA12 and FCN3, or fragment(s) thereof.

In embodiments, the method of the invention determines MMP9 or fragment(s) thereof and at least one further biomarker, preferably selected from the list comprising PAPP-A2, PAPP-A, NOTUM, MST1L, PSG3, ACTA, ADA12 and FCN3 or fragment(s) thereof.

In embodiments, the invention comprises determining a level of MMP9 or fragment(s) thereof and of NOTUM or fragments thereof, wherein the sample has preferably been isolated during the first trimester, such as week 9-13 or preferably during weeks 11-13 of gestation, and the PTB is preferably early PTB, more preferably very early PTB.

In embodiments, the invention comprises determining a level of MMP9 or fragment(s) thereof and of PAPP-A or fragment(s) thereof, wherein the sample has preferably been isolated during the first trimester, such as week 9-13 or preferably during weeks 11-13 of gestation, and the PTB is preferably early PTB, more preferably very early PTB.

In embodiments, the invention comprises determining a level of MMP9 or fragment(s) thereof and of PAPP-A2 or fragment(s) thereof, wherein the sample has preferably been isolated during the first trimester, such as week 9-13 or preferably during weeks 11-13 of gestation, and the PTB is preferably early PTB, more preferably very early PTB.

In embodiments, the invention comprises determining a level of MMP9 or fragment(s) thereof and of PAPP-A2 or fragment(s) thereof, wherein the sample has preferably been isolated during the second trimester, preferably during weeks 20-24 of gestation, and the PTB is preferably early PTB or very early PTB.

In embodiments, the invention comprises determining a level of MMP9 or fragment(s) thereof and of MST1L or fragment(s) thereof, wherein the sample has preferably been isolated during the second trimester, preferably during weeks 20-24 of gestation, and the PTB is preferably early PTB or very early PTB.

In embodiments, the invention comprises determining a level of MMP9 or fragment(s) thereof and of PSG3 or fragment(s) thereof, wherein the sample has preferably been isolated during the second trimester, preferably during weeks 20-24 of gestation, and the PTB is preferably early PTB or very early PTB.

In embodiments, the invention comprises determining a level of MMP9 or fragment(s) thereof and of PAPP-A2 or fragment(s) thereof, wherein the sample has preferably been isolated during the third trimester, preferably during weeks 30-34 of gestation, and the PTB is preferably early PTB or very early PTB.

In embodiments, the invention comprises determining a level of MMP9 or fragment(s) thereof and of ACTA or fragment(s) thereof, wherein the sample has preferably been isolated during the third trimester, preferably during weeks 30-34 of gestation, and the PTB is preferably early PTB or very early PTB.

In embodiments, the invention comprises determining a level of MMP9 or fragment(s) thereof and of ADA12 or fragment(s) thereof, wherein the sample has preferably been isolated during the third trimester, preferably during weeks 30-34 of gestation, and the PTB is preferably early PTB or very early PTB.

In embodiments, the invention comprises determining a level of MMP9 or fragment(s) thereof and of FCN3 or fragment(s) thereof, wherein the sample has preferably been isolated during the third trimester, preferably during weeks 30-34 of gestation, and the PTB is preferably early PTB or very early PTB.

In preferred embodiments, the one or more biomarkers comprise PAPP-A2 or fragment(s) thereof.

In embodiments, the levels PAPP-A2 or fragment(s) thereof is determined in a sample that has been isolated in the first, second or third trimester of pregnancy. In embodiments, the levels PAPP-A2 or fragment(s) thereof is determined in a sample that has been isolated in the second or preferably third trimester of pregnancy, wherein preferably no further biomarker is determined.

It was completely unexpected that in embodiments it is possible to predict all kinds of spontaneous PTB within the 2^{nd} and preferably 3^{rd} trimester, preferably in weeks 20-24 or 30-24, of gestation, respectively, using PAPP-A2 as a single biomarker or in combination with other biomarkers or clinical parameters. Furthermore, surprisingly PAPP-A2 in combination with other biomarkers can predict sPTB, in particular early PTB, in case of sample isolation in the first trimester, such as during weeks 9-13 or preferably 11-13.

In embodiments, the method of the invention determines PAPP-A2 or fragment(s) thereof as a single biomarker (meaning that no other biomarker is determined in the context of the method of the invention). In such embodiments, the sample may have been isolated during the second trimester, preferably week 20-24 of gestation, and PTB may be before week 37 or early PTB or very early PTB, preferably early PTB or very early PTB.

In embodiments, the method of the invention determines PAPP-A2 or fragment(s) thereof as a single biomarker and the sample has been isolated during the third trimester, preferably week 30-34 of gestation, and PTB may be before week 37 or early PTB, preferably early PTB.

In embodiments, the method of the invention determines PAPP-A2 or fragment(s) thereof and at least one further biomarker, preferably selected from the list comprising of MMP9, LTF, CGA, INHA, ACTA, NOTUM, PAPP-A, PSG3, MST1L, SHBG, sFlt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1, MMP8, or fragment(s) thereof. It was found herein that combining determination of PAPP-A2 with further biomarkers can increase the predictive value of the method.

In embodiments, the method of the invention determines PAPP-A2 or fragment(s) thereof and at least one further biomarker, preferably selected from the list comprising MMP9, LTF, CGA, INHA, ACTA, NOTUM, PAPP-A, PSG3, MST1L, SHBG, sFlt-1, ADA12 and FCN3, or fragment(s) thereof.

In embodiments, the method of the invention determines PAPP-A2 or fragment(s) thereof and at least one further biomarker, preferably selected from the list comprising MMP9, LFT, CGA, INHA, PAPP-A, SHBG, sFlt-1 and MST1L or fragment(s) thereof.

In embodiments, the invention comprises determining a level of PAPP-A2 or fragment(s) thereof and of MMP9 or fragments thereof, wherein the sample has preferably been isolated during the first trimester, such as week 9-13 or preferably during weeks 11-13 of gestation, and the PTB is preferably early PTB, more preferably very early PTB.

In embodiments, the invention comprises determining a level of PAPP-A2 or fragment(s) thereof and of LTF or fragment(s) thereof, wherein the sample has preferably been isolated during the first trimester, such as week 9-13 or preferably during weeks 11-13 of gestation, and the PTB is preferably early PTB, more preferably very early PTB.

In embodiments, the invention comprises determining a level of PAPP-A2 or fragment(s) thereof and of CGA or fragment(s) thereof, wherein the sample has preferably been isolated during the first trimester, such as week 9-13 or preferably during weeks 11-13 of gestation, and the PTB is preferably early PTB, more preferably very early PTB.

In embodiments, the invention comprises determining a level of PAPP-A2 or fragment(s) thereof and of MMP9 or fragment(s) thereof, wherein the sample has preferably been isolated during the second trimester, preferably during weeks 20-24 of gestation, and the PTB is preferably early PTB or very early PTB.

In embodiments, the invention comprises determining a level of PAPP-A2 or fragment(s) thereof and of INHA or fragment(s) thereof, wherein the sample has preferably been isolated during the second trimester, preferably during weeks 20-24 of gestation, and the PTB is preferably early PTB or very early PTB.

In embodiments, the invention comprises determining a level of PAPP-A2 or fragment(s) thereof and of CGA or fragment(s) thereof, wherein the sample has preferably been isolated during the second trimester, preferably during weeks 20-24 of gestation, and the PTB is preferably early PTB or very early PTB.

In embodiments, the invention comprises determining a level of PAPP-A2 or fragment(s) thereof and of PAPP-A or fragment(s) thereof, wherein the sample has preferably been isolated during the second trimester, preferably during weeks 20-24 of gestation, and the PTB is preferably early PTB or very early PTB.

In embodiments, the invention comprises determining a level of PAPP-A2 or fragment(s) thereof and of MMP9 or fragment(s) thereof, wherein the sample has preferably been isolated during the third trimester, preferably during weeks 30-34 of gestation, and the PTB is preferably early PTB or very early PTB.

In embodiments, the invention comprises determining a level of PAPP-A2 or fragment(s) thereof and of SHBG or fragment(s) thereof, wherein the sample has preferably been isolated during the third trimester, preferably during weeks 30-34 of gestation, and the PTB is preferably early PTB or very early PTB.

In embodiments, the invention comprises determining a level of PAPP-A2 or fragment(s) thereof and of PAPP-A or fragment(s) thereof, wherein the sample has preferably been isolated during the third trimester, preferably during weeks 30-34 of gestation, and the PTB is preferably early PTB or very early PTB.

In embodiments, the invention comprises determining a level of PAPP-A2 or fragment(s) thereof and of sFlt-1 or fragment(s) thereof, wherein the sample has preferably been isolated during the third trimester, preferably during weeks 30-34 of gestation, and the PTB is preferably early PTB or very early PTB.

In embodiments, the invention comprises determining a level of PAPP-A2 or fragment(s) thereof and of MST1L or fragment(s) thereof, wherein the sample has preferably been isolated during the third trimester, preferably during weeks 30-34 of gestation, and the PTB is preferably early PTB or very early PTB.

In embodiments, the method additionally comprises treating a subject with an increased risk of PTB, preferably by applying PTB preventive measures.

In embodiments, the method additionally comprises treating a subject with an increased risk of PTB, preferably by applying PTB preventive and/or therapeutic measures.

Depending on the result of the method of the present invention, embodiments of the method may comprise subsequent therapeutic decisions and/or therapeutic actions. Such therapeutic decisions may include the initiation, change or modification of medical treatment of the pregnant subject. Such treatment may be directed to reducing the risk of or preventing PTB or to ameliorate the consequences of PTB. Any therapy, medical treatment or therapeutic action disclosed herein can be employed in the context of the method of the invention as a subsequent therapeutic decision or therapeutic action.

On the other hand, if the result of the method of the present invention is indicative of the absence of risk of developing PTB, no specific treatment measures with respect to PTB may be required.

The invention further relates to methods of treatment for PTB, wherein the pregnant subject to be treated is identified, stratified, monitored, prognosed, diagnosed or otherwise assessed using the methods described herein. Suitable treatments for the methods are disclosed herein. The present invention is therefore particularly advantageous in identifying pregnant subjects with increased risk of PTB and initiating preventative or risk-reducing treatments, or initiating treatments to address the presence of PTB.

In embodiments of the invention, a method as described herein is performed at least two, preferably three or more times, preferably using samples that have been isolated from the pregnant subject over the first, second and/or third trimester of pregnancy. In such embodiments, the method of the invention may be repeated in the context of a workflow in case the previously determined level of the one or more biomarkers indicated the presence of a subsequent PTB. In that case the pregnant subject may be determined to be at risk of PTB and is assigned for repeating the method of the present invention at a later time point during pregnancy to assess whether the subject can still be considered to be at risk of PTB.

In the context of such a work flow method of the invention comprising repeated performance of the method of the invention over the course of a pregnancy in case the a subject is categorized as being at risk of PTB, the time point of first performing of the method of the invention is not limiting. The method may be performed for the first time during the first, second or third trimester of pregnancy. However, once a determined biomarker level indicated the presence of a subsequent adverse event leading to the categorization of the subject being at risk of PTB, the method may lead to initiation of preventive measures of PTB as described herein. Furthermore, due to the determined increased risk for PTB of the subject, in embodiments the method will be repeated after a certain period of time, and/or after preventive measures have been applied, to determine whether the determined biomarker levels still indicate the presence of a subsequent PTB. In embodiments, the method of the invention may be repeated multiple times over the course of the pregnancy as long as the subject is categorized as being at risk of PTB due to a determined increased biomarker level as compared to a reference level.

In embodiments, the method of the invention may be performed at several time points of gestation, potentially as part of a routine screening of pregnant subjects, irrespective of whether a previous measurement indicated an increased risk. In embodiments, monitoring the one or more biomarkers of the invention at several time points over the course of pregnancy may be advantageous, for example when a subject is suspected of being at risk of PTB. In embodiments, repeated measurement/determing of the one or more biomarkers of the invention may be performed also when a first or earlier measurement was not indicative of an increased risk of PTB.

Preferably, determining an increased biomarker level at a single time point is sufficient for categorizing a subject as being at risk of PTB. Conversely, in certain embodiments, for categorizing a subject that has previously been determined to be at risk of PTB as no longer being at risk of PTB, it can be required to determine a biomarker level that is indicative of the absence of PTB at least at one later time point, preferably at least two or three later time points, preferably consecutively.

In a further aspect, the present invention relates to a kit for carrying out a method for the diagnosis, prognosis, prediction, risk assessment and/or risk stratification of preterm birth (PTB) in a pregnant subject as disclosed herein, the kit comprising:
detection reagents for determining a level of MMP9 or fragment(s) thereof and/or PAPP-A2 or fragment(s) thereof in a sample from a pregnant subject,
and optionally, detection reagents for determining a level of at least one additional biomarker or fragment(s) thereof, preferably selected from the group comprising or consisting of LTF, CGA, INHA, ACTA, NOTUM, PAPP-A, PSG3, MST1L, SHBG, sFIt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1 and MMP8 in a sample from a patient, and
reference data, or means to obtain reference data, for the risk of whether PTB in a pregnant subject will occur, wherein the reference data comprise reference levels for MMP9 and/or PAPP-A2, and optionally additionally reference levels for said at least one additional parameter or biomarker,
wherein said reference data is preferably stored on a computer readable medium and/or employed in the form of a computer executable code, such as an algorithm, configured for comparing the determined levels of MMP9 or fragment(s) thereof and/or PAPP-A2 or fragment(s) thereof and optionally of the additional biomarker or fragment(s) thereof, with the provided reference levels.

In one embodiment, the present invention relates to a kit for carrying out a method for the diagnosis, prognosis, prediction, risk assessment and/or risk stratification of preterm birth (PTB) in a pregnant subject as disclosed herein, the kit comprising:
detection reagents for determining a level of MMP9 or fragment(s) thereof in a sample from a pregnant subject,
and optionally, detection reagents for determining a level of at least one additional biomarker or fragment(s) thereof, preferably selected from the group comprising or consisting of PAPP-A2, LTF, CGA, INHA, ACTA, NOTUM, PAPP-A, PSG3, MST1L, SHBG, sFlt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1 and MMP8 in a sample from a patient, and
reference data, or means to obtain reference data, for the risk of whether PTB in a pregnant subject will occur, wherein the reference data comprise reference levels for MMP9, and optionally additionally reference levels for said at least one additional parameter or biomarker,
wherein said reference data is preferably stored on a computer readable medium and/or employed in the form of a computer executable code, such as an algorithm, configured for comparing the determined levels of or fragment(s) thereof and optionally of the additional biomarker or fragment(s) thereof, with the provided reference levels.

In one embodiment, the present invention relates to a kit for carrying out a method for the diagnosis, prognosis, prediction, risk assessment and/or risk stratification of preterm birth (PTB) in a pregnant subject as disclosed herein, the kit comprising:
detection reagents for determining a level of PAPP-A2 or fragment(s) thereof in a sample from a pregnant subject,
and optionally, detection reagents for determining a level of at least one additional biomarker or fragment(s) thereof, preferably selected from the group comprising or consisting of MMP9, LTF, CGA, INHA, ACTA, NOTUM, PAPP-A, PSG3, MST1L, SHBG, sFIt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1 and MMP8 in a sample from a patient, and
reference data, or means to obtain reference data, for the risk of whether PTB in a pregnant subject will occur, wherein the reference data comprise reference levels for PAPP-A2, and optionally additionally reference levels for said at least one additional parameter or biomarker,
wherein said reference data is preferably stored on a computer readable medium and/or employed in the form of a computer executable code, such as an algorithm, configured for comparing the determined levels of PAPP-A2 or fragment(s) thereof and optionally of the additional biomarker or fragment(s) thereof, with the provided reference levels.

In embodiments, the kit of the invention may comprise components to stabilize the testing sytem and/or that garantee reproducibility, such as calibrators, puffer, standard solutions, testing solutions and software systems.

As explained herein, in the context of the invention, the determined levels of the biomarkers of the invention, in particular MMP9 and/or PAPP-A2 or fragment(s) thereof, are compared to reference levels that are determined from a control group of subjects without prenatal disorder or condition or PTB at the corresponding time point of pregnancy. In the context of the invention, the determined levels of the biomarkers of the invention, in particular MMP9 or fragment(s) thereof, are compared to reference levels that are determined from a control group of subjects without prenatal disorder or condition or PTB at the corresponding time point of pregnancy. In the context of the invention, the determined levels of the biomarkers of the invention, in particular PAPP-A2 or fragment(s) thereof, are compared to reference levels that are determined from a control group of subjects without prenatal disorder or condition or PTB at the corresponding time point of pregnancy. Accordingly, the reference data of the invention can comprise multiple reference levels for each biomarker for multiple different time points over the course of a pregnancies, such as reference levels for each trimester of pregnancy, for each week of pregnancy and/or each day of pregnancy. In embodiments, the reference data may comprise reference levels for each employed biomarker for the first, second and/or trimester, for weeks 11-13, 20-24 and/or 30-34 of pregnancy, and/or for week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and/or 37 of pregnancy.

Using the reference levels of the biomarkers at the respective time points of pregnancy, the comparison between the determined level and the reference level may be performed in different ways. In embodiments, a FC or a MoM may be calculated, wherein a threshold value for FC or MoM for a respective biomarker at a respective time point may be included in the reference data. In embodiments, the reference data may in addition or alternatively comprise absolute threshold concentrations of the biomarker for specific pregnancy time points.

All features and embodiments that have been disclosed and explained herein in the context of the method of the invention are herewith also disclosed in the context of the kit of the invention, and vice versa.

Determining of the levels of the various biomarkers in the context of the present invention can be performed according to any known method described in the art or and any method that will be developed in the future. The method and kit of the present invention is not limited to specific concentrations as determined by a specific method, but is based on the surprising finding that elevated/increased levels of biomarkers, in particular MMP9 and/or PAPP-A2, in a sample from a pregnant subject as compared to reference levels in a suitable control group as described herein are indicative of the presence of subsequent PTB, whereas levels that are not increased are indicative of the absence of PTB.

In embodiments, the determined level of the one or more biomarkers biomarkers correlates an increased risk of PTB.

Therefore, the method of determining the level of a respective biomarker in the context of the invention is not of primary relevance, as long as the determined level is comparable to the reference level, which may be predetermined and may be provided for example on a data storage device and may be incorporated in a diagnostic algorithm.

For example, the levels of the biomarker disclosed herein may be determined by ELISA or similar methods, by the use of automated systems such as the B-R-A-H-M-S™ KRYPTOR™ analyzers or by mass spectrometry, as described in the examples below. In embodiments, CGA, INHA, PAPP-A and sFIt-1 serum concentrations may be determined using the B-R-A-H-M-S™ KRYPTOR™ instrument. The levels of MMP9, LTF, NOTUM, PSG3, MST1L, SHBG, ADA12 and FCN3 may be determined in the serum samples by Selected Reaction Monitoring (SRM) assays. SRM assays is the targeted measurement of specific peptides derived from the biomarkers by LC-MS/MS technology. In embodiments, PAPP-A2, and Activin A serum concentrations can be determined using commercially available ELISA Kits. Depending on the method of the determining a respective biomarker, the determined concentration unit may differ. Accordingly, the specific values disclosed herein are intended to also read on the corresponding values determined by other methods.

In embodiments of the methods described herein the level of MMP9 and/or PAPP-A2 or fragment(s) thereof and optionally additionally other biomarkers as disclosed herein is determined using a method selected from the group consisting of mass spectrometry (MS), luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats such as for instance immunochromatographic strip tests, rare cryptate assay, and automated systems/analyzers.

In embodiments of the methods described herein the level of MMP9 or fragment(s) thereof and optionally additionally other biomarkers as disclosed herein is determined using a method selected from the group consisting of mass spectrometry (MS), luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats such as for instance immunochromatographic strip tests, rare cryptate assay, and automated systems/analyzers.

In embodiments of the methods described herein the level of PAPP-A2 or fragment(s) thereof and optionally additionally other biomarkers as disclosed herein is determined using a method selected from the group consisting of mass spectrometry (MS), luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats such as for instance immunochromatographic strip tests, rare cryptate assay, and automated systems/analyzers.

The method according to the present invention can furthermore be embodied as a homogeneous method, wherein the sandwich complexes formed by the antibody/antibodies and the marker, e.g., the MMP9 or PAPP-A2 or fragments thereof, which is to be detected remains suspended in the liquid phase. In this case it is preferred, that when two antibodies are used, both antibodies are labelled with parts of a detection system, which leads to generation of a signal or triggering of a signal if both antibodies are integrated into a single sandwich.

Such techniques are to be embodied in particular as fluorescence enhancing or fluorescence quenching detection methods. A particularly preferred aspect relates to the use of detection reagents which are to be used pair-wise, such as for example the ones which are described in U.S. Pat. No. 4,882,733 A, EP-B1 0 180 492 or EP-B1 0 539 477 and the prior art cited therein. In this way, measurements in which only reaction products comprising both labelling components in a single immune-complex directly in the reaction mixture are detected, become possible.

For example, such technologies are offered under the brand names TRACE™ (Time Resolved Amplified Cryptate Emission) or KRYPTOR™, implementing the teachings of the above-cited applications. Therefore, in particular preferred aspects, a diagnostic device is used to carry out the herein provided method. For example, the level of the MMP9 and/or PAPP-A2 protein or a fragment thereof, and/or the level of any further marker of the herein provided method are determined. In particular preferred aspects, the diagnostic device is KRYPTOR™.

In one embodiment of the method described herein the method is an immunoassay and wherein the assay is performed in homogeneous phase or in heterogeneous phase.

In further embodiments of the method described herein, the method additionally comprises a molecular analysis of a sample from said pregnant subject. The sample used for the molecular analysis for risk assessment of PTB preferably is a blood sample. In a preferred embodiment the molecular analysis is a method aiming to detect one or more biomolecules associated with PTB.

Said one or more biomolecule may be a nucleic acid, protein, sugar, carbohydrades, lipid and or a combination thereof such as glycosylated protein, preferably a nucleic acid. Methods of molecular analysis are known to the person skilled in the art and are comprised by the method of the present invention.

The methods of the invention can be performed in combination with mass spectrometry to detect relevant biomarkers of the method of the invention as for example disclosed in patent U.S. Pat. No. 9,074,236. Furthermore, in embodiments the methods of the invention can be performed in combination with further diagnostic procedures, such as x-ray analysis, ultrasound examination, CT scanning or other diagnostic imaging techniques.

In one embodiment of the method described herein, the method additionally comprises comparing the determined level of the one or more biomarkers comprising at least one of MMP9 and/or PAPP-A2 or fragment(s) thereof to a reference level, which may be threshold value, a threshold concentration, a population mean and/or a population median of the respective biomarker at the respective time point in subjects with a normal pregnancy, in particular without PTB, wherein said comparing is carried out in a computer processor using computer executable code.

In one embodiment of the method described herein, the method additionally comprises comparing the determined level of the one or more biomarkers comprising MMP9 or fragment(s) thereof to a reference level, which may be threshold value, a threshold concentration, a population mean and/or a population median of the respective biomarker at the respective time point in subjects with a normal pregnancy, in particular without PTB, wherein said comparing is carried out in a computer processor using computer executable code.

In one embodiment of the method described herein, the method additionally comprises comparing the determined level of the one or more biomarkers comprising PAPP-A2 or fragment(s) thereof to a reference level, which may be threshold value, a threshold concentration, a population mean and/or a population median of the respective biomarker at the respective time point in subjects with a normal pregnancy, in particular without PTB, wherein said comparing is carried out in a computer processor using computer executable code.

The methods and kits of the present invention may in part be computer-implemented. For example, the step of comparing the detected level of a marker, e.g. the MMP9 or fragments thereof and/or PAPP-A2 or fragement(s) thereof, with a reference level can be performed in a computer system. In one embodiment the step of comparing the detected level of a marker, e.g. the MMP9 or fragments thereof, with a reference level can be performed in a computer system. In one embodiment the step of comparing the detected level of a marker, e.g. PAPP-A2 or fragement(s) thereof, with a reference level can be performed in a computer system. In the computer-system, the determined level of the marker(s) can be combined with other marker levels and/or parameters of the subject in order to calculate a score, which is indicative for the diagnosis, prognosis, prediction, risk assessment and/or risk stratification of PTB. For example, the determined values may be entered (either manually by a health professional or automatically from the device(s) in which the respective marker level(s) has/have been determined) into the computer-system. The computer-system can be directly at the point-of-care or it can be at a remote location connected via a computer network (e.g. via the internet, or specialized medical cloud-systems, optionally combinable with other IT-systems or platforms such as hospital information systems (HIS)). Typically, the computer-system will store the values (e.g. marker level or parameters such as age, blood pressure, weight, sex, etc. or clinical scoring systems or clinical assessments such as cervical length assessment, BMI etc.) on a computer-readable medium and calculate the score based-on pre-defined and/or pre-stored reference levels or reference values. The resulting score will be displayed and/or printed for the user (typically a health professional such as a physician). Alternatively or in addition, the associated prognosis, diagnosis, assessment, treatment guidance, patient management guidance or stratification will be displayed and/or printed for the user (typically a health professional such as a physician).

In one embodiment of the invention, a software system can be employed, in which a machine learning algorithm is evident, preferably to identify pregnant subjects at risk of PTB using data from electronic health records (EHRs) including determined biomarker levels. A machine learning approach can be trained on a random forest classifier using EHR data (such as labs, biomarker expression, vitals, and demographics) from pregnant subjects. Machine learning is a type of artificial intelligence that provides computers with the ability to learn complex patterns in data without being explicitly programmed, unlike simpler rule-based systems. Earlier studies have used electronic health record data to trigger alerts to detect clinical deterioration in general. In one embodiment of the invention the processing of MMP9 and/or PAPP-A2 levels may be incorporated into appropriate software for comparison to existing data sets, for example MMP9 and/or PAPP-A2 levels may also be processed in machine learning software to assist in diagnosing or prognosing the occurrence of PTB.

In one embodiment of the invention the processing of MMP9 levels may be incorporated into appropriate software for comparison to existing data sets, for example MMP9 levels may also be processed in machine learning software to assist in diagnosing or prognosing the occurrence of PTB.

In one embodiment of the invention the processing of PAPP-A2 levels may be incorporated into appropriate software for comparison to existing data sets, for example PAPP-A2 levels may also be processed in machine learning software to assist in diagnosing or prognosing the occurrence of PTB.

The combined employment of MMP9 and/or PAPP-A2 in combination with each other or in combination with further biomarkers may be realized either in a single multiplex assay, or in two separate assays conducted on a sample form the pregnant subject. In one embodiment the employment of MMP9 in combination with further biomarkers may be realized either in a single multiplex assay, or in two separate assays conducted on a sample form the pregnant subject. In one embodiment the employment of PAPP-A2 in combination with further biomarkers may be realized either in a single multiplex assay, or in two separate assays conducted on a sample form the pregnant subject. The sample may relate to the same sample, or to different samples. The assay employed for the detection and determination of the different biomarkers may be the same or different, for example an immunoassay may be employed for the determination of one of the above markers, and a MS assay may be used for another. More detailed descriptions of suitable assays are provided below.

Reference levels of the biomarkers used in the context of the method of the invention may be determined by previously described methods. For example, methods are known to a skilled person for using the coefficient of variation in assessing variability of quantitative assays in order to establish reference values and/or cut-offs (George F. Reed et al., Clin Diagn Lab Immunol.2002; 9 (6): 1235-1239).

Additionally, functional assay sensitivity can be determined in order to indicate statistically significant values for use as reference levels or cut-offs according to established techniques. Laboratories are capable of independently establishing an assay's functional sensitivity by a clinically relevant protocol. “Functional sensitivity” can be considered as the concentration that results in a coefficient of variation (CV) of 20% (or some other predetermined % CV), and is thus a measure of an assay's precision at low analyte levels. The CV is therefore a standardization of the standard deviation (SD) that allows comparison of variability estimates regardless of the magnitude of analyte concentration, at least throughout most of the working range of the assay.

Furthermore, methods based on ROC analysis can be used to determine statistically significant differences between two clinical patient groups. Receiver Operating Characteristic (ROC) curves measure the sorting efficiency of the model's fitted probabilities to sort the response levels. ROC curves can also aid in setting criterion points in diagnostic tests. The higher the curve from the diagonal, the better the fit. If the logistic fit has more than two response levels, it produces a generalized ROC curve. In such a plot, there is a curve for each response level, which is the ROC curve of that level versus all other levels. Software capable of enabling this kind of analysis in order to establish suitable reference levels and cut-offs is available, for example JMP 12, JMP 13, Statistical Discovery, from SAS.

Population averages levels may also be used as reference values, for example mean or median population values, whereby pregnant subjects may be compared to a control population for a given time point of the pregnancy, wherein the control group preferably comprises more than 10, 20, 30, 40, 50 or more subjects.

The invention relates further to a method for identifying subjects at risk of preterm birth (PTB) and treating said subjects, the method comprising:
(a) diagnosis, prognosis, prediction, risk assessment and/or risk stratification of preterm birth (PTB) in a pregnant subject, comprising:
   determining a level of metallopeptidase 9 (MMP9) or fragment(s) thereof in a sample that has been isolated from said pregnant subject,
   wherein the level of MMP9 in the sample is indicative of the presence of a subsequent PTB, and
(b) administering to said subject a treatment for preterm birth (PTB).

The invention relates further to a method for identifying subjects at risk of preterm birth (PTB) and treating said subjects, the method comprising:
(a) diagnosis, prognosis, prediction, risk assessment and/or risk stratification of preterm birth (PTB) in a pregnant subject, comprising:
   determining a level of Pappalysin-2 (PAPP-A2) or fragment(s) thereof in a sample that has been isolated from said pregnant subject,
   wherein the level of PAPP-A2 in the sample is indicative of the presence of a subsequent PTB, and
(b) administering to said subject a treatment for preterm birth (PTB).

The invention relates further to a method for detecting metallopeptidase 9 (MMP9) or fragment(s) thereof in a sample from a subject, the method comprising:
providing a sample of a subject, preferably a blood sample or sample derived from a blood sample, having a complex comprising at least one binder to MMP9 or fragment(s) thereof;
wherein the sample has a level of MMP9 that is above a threshold value, such as any threshold disclosed herein, preferably a population mean and/or a population median of MMP9 levels from a normal pregnancy at any given time point in the respective patient population.

The invention relates further to a method for detecting Pappalysin-2 (PAPP-A2) or fragment(s) thereof in a sample from a subject, the method comprising:
providing a sample of a subject, preferably a blood sample or sample derived from a blood sample, having a complex comprising at least one binder to Pappalysin-2 (PAPP-A2) or fragment(s) thereof;
wherein the second sample has a level of PAPP-A2 that is above a threshold value, such as any threshold disclosed herein, preferably a population mean and/or a population median of PAPP-A2 levels from a normal pregnancy at any given time point in the respective patient population.

The invention relates further to a method for treating and/or reducing the risk of preterm birth (PTB), or for administering to a subject a treatment for preterm birth (PTB), the method comprising:
administering to a subject a treatment for preterm birth (PTB),
wherein said subject has been determined to have, in a bodily fluid sample of the subject, preferably a blood sample or sample derived from a blood sample,
a level of metallopeptidase 9 (MMP9) or fragment(s) thereof that is above a threshold value, such as any threshold disclosed herein, preferably a population mean and/or a population median of MMP9 levels from a normal pregnancy at any given time point in the respective patient population, and/or
a level of Pappalysin-2 (PAPP-A2) or fragment(s) thereof that is above a threshold value, such as any threshold disclosed herein, preferably a population mean and/or a population median of PAPP-A2 levels from a normal pregnancy at any given time point in the respective patient population.

Aspects, embodiments and features of the invention described herein are considered to be disclosed with respect to each and every other aspect, embodiment and feature of the disclosure, such that for example features characterizing the method, may be employed to characterize the kit and vice-versa, and for example features characterizing the method referring to MMP9, may be employed to characterize the method referring to PAPP-A2, and vice versa.

### DETAILED DESCRIPTION OF THE INVENTION

The present invention is based on the surprising finding that PAPP-A2 and/or MMP9 are predictive biomarkers for preterm birth throughout pregnancy, which enable risk assessment of PTB over the entire course of a pregnancy, i.e. through all three trimesters of pregnancy. In particular, the invention enables diagnosis, prognosis, prediction, risk assessment and/or risk stratification of preterm birth (PTB) in a pregnant subject.

As used herein, the terms “patient” or “subject” are used interchangeably and relate to a vertebrate. In the context of the present invention, the term “subject” includes both humans and animals, particularly mammals, and other organisms. Preferably, the subject is a human subject, in particular a female human subject.

The term “pregnant” and “pregnancy”, herein also referred to as gestation, is the period during which one or more offspring develops inside a female subject, i.e. a woman. Childbirth typically occurs around 40 weeks from the start of the last menstrual period (LMP). This is just over nine months (gestational age)—where each month averages 31 days. When using fertilization age it is about 38 weeks. An embryo is the developing offspring during the first eight weeks following fertilization, (ten weeks' gestational age) after which, the term fetus is used until birth.

The chronology of pregnancy is, unless otherwise specified, generally given as gestational age, where the starting point is the beginning of the woman's last menstrual period (LMP), or the corresponding age of the gestation as estimated by a more accurate method if available. Sometimes, timing may also use the fertilization age which is the age of the embryo.

Pregnancy is divided into three trimesters, each lasting for approximately 3 months. The first trimester includes conception, which is when the sperm fertilizes the egg. The fertilized egg then travels down the Fallopian tube and attaches to the inside of the uterus, where it begins to form the embryo and placenta. During the first trimester, the possibility of miscarriage (natural death of embryo or fetus) is at its highest. The exact length of each trimester can vary between sources. The first trimester begins with the start of gestational age as described above, that is, 0 weeks+0 days of gestational age (GA). It ends at week 13 (13 weeks+6 days of GA).

If not explicitly stated otherwise, the time points of gestation (gestational age), such as an indicated gestational week, as referred to hereis is the gestational age calculated from the last menstrual period.

The second trimester is defined as starting, at the beginning of week 14 (14 weeks+0 days of GA). It ends at the end of week 26 (26 weeks+6 days of GA). Around the middle of the second trimester, movement of the fetus may be felt.

The third trimester is defined as starting, at the beginning of week 27 (27 weeks+0 days of GA)[32]. It lasts until childbirth.

At 28 weeks, more than 90% of babies can survive outside of the uterus if provided with high-quality medical care, though babies born at this time will likely experience serious health complications such as heart and respiratory problems and long-term intellectual and developmental disabilities.

In the ideal childbirth labor begins on its own when a woman is “at term”. Babies born before 37 weeks are “preterm” and at higher risk of health problems such as cerebral palsy. Babies born between weeks 37 and 39 are considered “early term” while those born between weeks 39 and 41 are considered “full term”. Babies born between weeks 41 and 42 weeks are considered “late term” while after 42 weeks they are considered “post term”. Delivery before 39 weeks by labor induction or caesarean section is not recommended unless required for other medical reasons.

Prenatal care improves pregnancy outcomes. Prenatal care may include taking extra folic acid, avoiding drugs, tobacco smoking, and alcohol, taking regular exercise, having blood tests, and regular physical examinations. In the context of the present invention, prenatal care measures are included when referring to (preventive) treatment options of PTB that may be indicated, applied or intensified upon determining an increased risk of PTB in a pregnant subject.

Complications of pregnancy that can be assessed as clinical parameters in the context of the invention may include disorders of high blood pressure, gestational diabetes, iron-deficiency anemia, and severe nausea and vomiting.

As used herein, preterm birth (PTB), also known as premature birth, is the birth of a baby at fewer than 37 weeks gestational age. Very early preterm birth is before 32 weeks, early preterm birth occurs between 32-34 weeks, late preterm birth is between 34-36 weeks' gestation. Preterm born babies are also referred to as premature babies or colloquially preemies or premmies. WHO has published a slightly deviating categorization wherein extremely preterm birth is less than 28 weeks, very preterm birth is 28 to 32 weeks, and moderate to late preterm is 32 to 37 weeks (https://www.who.int/news-room/fact-sheets/detail/preterm-birth).

Symptoms of preterm labor include uterine contractions which occur more often than every ten minutes and/or the leaking of fluid from the vagina before 37 weeks.

As used herein, the terms symptoms and signs of a condition/disorder are used interchangeably.

Premature infants are at greater risk for cerebral palsy, delays in development, hearing problems and problems with their vision. The earlier a baby is born, the greater these risks will be.

The cause of spontaneous preterm birth is often not known. Risk factors, such as clinical parameters that may be assessed in the context of the invention, include diabetes, high blood pressure, multiple gestation (being pregnant with more than one baby), being either obese or underweight, vaginal infections, air pollution exposure, tobacco smoking, and psychological stress.

Preterm birth may be prevented in those at risk if the hormone progesterone is taken during pregnancy. Accordingly, progesterone administration may be a preventive/therapeutic measure that may be indicated or started as a result of determining an increased risk of PTB using the method of the invention. Another preventive/therapeutic treatment measure of PTB is bed, although its effectiveness is controversial. Further preventive/therapeutic treatment measures for subjects at risk of PTB include corticosteroid treatment, cervical cerclage and a number of medications known to the skilled person, such as nifedipine, which may delivery.

It is estimated that at least 75% of preterm infants would survive with appropriate treatment, and the survival rate is highest among the infants born the latest in gestation. Once the baby is born, care includes keeping the baby warm through skin-to-skin contact or incubation, supporting breastfeeding and/or formula feeding, treating infections, and supporting breathing. Preterm babies sometimes require intubation.

Preterm birth is the most common cause of death among infants worldwide. About 15 million babies are preterm each year (5% to 18% of all deliveries). Late preterm birth accounts for 75% of all preterm births. This rate is inconsistent across countries. In the United Kingdom 7.9% of babies are born pre-term and in the United States 12.3% of all births are before 37 weeks gestation. Approximately 0.5% of births are extremely early periviable births (20-25 weeks of gestation), and these account for most of the deaths. In many countries, rates of premature births have increased between the 1990s and 2010s. Complications from preterm births resulted in 0.81 million deaths in 2015, down from 1.57 million in 1990. The chance of survival at 22 weeks is about 6%, while at 23 weeks it is 26%, 24 weeks 55% and 25 weeks about 72%. The chances of survival without any long-term difficulties are lower.

PTB due to preterm labor with cervical dilation or preterm rupture of membranes is classified as “spontaneous” PTB (sPTB). Labor which is induced or in which the infant is delivered by cesarean section for maternal or fetal illness is classified as “indicated” preterm birth. This splitting of PTB phenotypes is one attempt to separate distinct pathophysiologic pathways and patients who may benefit from different prediction, prevention, and treatment strategies. The present invention preferably relates to sPTB.

In embodiments, the method of the invention is performed in a pregnant subject with an increased risk of PTB as determined by known risk factors of PTB as described herein.

Signs and symptoms of PTB include four or more uterine contractions in one hour. In contrast to false labour, true labor is accompanied by cervical dilatation and effacement. Also, vaginal bleeding in the third trimester, heavy pressure in the pelvis, or abdominal or back pain could be indicators that a preterm birth is about to occur. A watery discharge from the vagina may indicate premature rupture of the membranes that surround the baby. While the rupture of the membranes may not be followed by labor, usually delivery is indicated as infection (chorioamnionitis) is a serious threat to both fetus and mother. In some cases, the cervix dilates prematurely without pain or perceived contractions, so that the mother may not have warning signs until very late in the birthing process.

Risk factors of PTB that may be assessed in the context of the present invention are described in the following. The exact cause of spontaneous preterm birth is difficult to determine and it may be caused by many different factors at the same time as labor is a complex process. At least four different pathways have been identified that can result in preterm birth and have considerable evidence: precocious fetal endocrine activation, uterine overdistension (placental abruption), decidual bleeding, and intrauterine inflammation or infection. Identifying women at high risk of giving birth early would enable the health services to provide specialized care for these women and their babies, for example a hospital with a special care baby unit such as a neonatal intensive care unit (NICU). In some instances, it may be possible to delay the birth. Risk scoring systems have been suggested as an approach to identify those at higher risk, however, it is unclear whether the use of risk scoring systems of the state of the art for identifying mothers would prolong pregnancy and reduce the numbers of preterm births or not.

Risk factors in the mother have been identified that are linked to a higher risk of a preterm birth. These include age, high and low body mass index (BMI), length of time between pregnancies, previous spontaneous (i.e., miscarriage) or surgical abortions, unintended pregnancies, untreated or undiagnosed celiac disease, fertility difficulties, heat exposure and genetic variables. Stressful conditions, hard labor, and long working hours are also probably linked to preterm birth. Obesity does not directly lead to preterm birth, however, it is associated with diabetes and hypertension which are risk factors by themselves. To some degree those individuals may have underlying conditions (i.e. uterine malformation, hypertension, diabetes) that persist. Couples who have tried more than 1 year versus those who have tried less than 1 year before achieving a spontaneous conception have also an increased risk of PTB. Pregnancies after in vitro fertilization (IVF) confers a greater risk of preterm birth than spontaneous conceptions after more than 1 year of trying.

Certain ethnicities may have a higher risk of PTB. For example, in the U.S. and the UK, Black women have preterm birth rates of 15-18%, more than double than that of the white population. Many Black women have higher preterm birth rates due to multiple factors but the most common is high amounts of chronic stress, which can eventually lead to premature birth. Filipinos are also at high risk of premature birth, and it is believed that nearly 11-15% of Filipinos born in the U.S. (compared to other Asians at 7.6% and whites at 7.8%) are premature. Filipinos being a big risk factor is evidenced with the Philippines being the eighth-highest ranking in the world for preterm births, the only non-African country in the top 10. Genetic make-up is a factor in the causality of preterm birth. Genetics has been a big factor into why Filipinos have a high risk of premature birth as the Filipinos have a large prevalence of mutations that help them be predisposed to premature births. An intra- and transgenerational increase in the risk of preterm delivery has been demonstrated. No single gene has been identified.

Marital status is associated with risk for preterm birth. A study of 25,373 pregnancies in Finland revealed that unmarried mothers had more preterm deliveries than married mothers (P=0.001). A study in Quebec of 720,586 births from 1990 to 1997 revealed less risk of preterm birth for infants with legally married mothers compared with those with common-law wed or unwed parents.

Medications during pregnancy, living conditions, air pollution, smoking, illicit drugs or alcohol, infection, or physical trauma may also cause a preterm birth.

The use of fertility medication that stimulates the ovary to release multiple eggs and of IVF with embryo transfer of multiple embryos has been implicated as a risk factor for preterm birth.

Certain medical conditions in the pregnant mother may also increase the risk of preterm birth. Some women have anatomical problems that prevent the baby from being carried to term. These include a weak or short cervix (the strongest predictor of premature birth). Women with vaginal bleeding during pregnancy are at higher risk for preterm birth. While bleeding in the third trimester may be a sign of placenta previa or placental abruption—conditions that occur frequently preterm—even earlier bleeding that is not caused by these conditions is linked to a higher preterm birth rate. Women with abnormal amounts of amniotic fluid, whether too much (polyhydramnios) or too little (oligohydramnios), are also at risk. Anxiety and depression have been linked as risk factors for preterm birth.

The use of tobacco, cocaine, and excessive alcohol during pregnancy increases the chance of preterm delivery. Tobacco is the most commonly used drug during pregnancy and contributes significantly to low birth weight delivery. Babies with birth defects are at higher risk of being born preterm. Passive smoking and/or smoking before the pregnancy influences the probability of a preterm birth. Presence of anti-thyroid antibodies is associated with an increased risk preterm birth. Intimate violence against the mother is another risk factor for preterm birth. Physical trauma may case a preterm birth.

The frequency of infection in preterm birth is inversely related to the gestational age. Mycoplasma genitalium infection is associated with increased risk of preterm birth, and spontaneous abortion. Fetal infection is linked to preterm birth and to significant long-term handicap including cerebral palsy. Bacterial vaginosis before or during pregnancy may affect the decidual inflammatory response that leads to preterm birth. The condition known as aerobic vaginitis can be a serious risk factor for preterm labor.

Untreated yeast infections are associated with preterm birth. Prophylactic antibiotics (given to prevent infection) in the second and third trimester of pregnancy (13-42 weeks of pregnancy) found a reduction in the number of preterm births in women with bacterial vaginosis. Accordingly, antibiotic treatment can be considered a preventive/therapeutic intervention for PTB in the context of the invention. Antibiotics also reduced the number of waters breaking before labor in full-term pregnancies, reduced the risk of infection of the lining of the womb after delivery (endometritis), and rates of gonococcal infection.

A number of maternal bacterial infections are associated with preterm birth including pyelonephritis, asymptomatic bacteriuria, pneumonia, and appendicitis.

It was found that preterm births happened less for pregnant women who had routine testing for low genital tract infections than for women who only had testing when they showed symptoms of low genital tract infections. The women being routinely tested also gave birth to fewer babies with a low birth weight. Periodontal disease is also linked to preterm birth.

It is believed that there is a maternal genetic component in preterm birth.

The method of the invention may be combined with or performed in addition to or instead of known diagnostic tests or appraoche for PTB.

Placental alpha microglobulin-1 (PAMG-1) is a human protein that was first isolated from amniotic fluid. PAMG-1 is an important biomarker for the detection of premature rupture of fetal membrane (PROM). The high concentration of PAMG-1 is in amniotic fluid, which means that it can be used to detect if this fluid is present in the cervico-vaginal discharge of pregnant women. Placental alpha microglobulin-1 (PAMG-1) has been the subject of several investigations evaluating its ability to predict imminent spontaneous preterm birth in women with signs, symptoms, or complaints suggestive of preterm labor. Comparison to fetal fibronectin testing and cervical length measurement via transvaginal ultrasound, the test for PAMG-1 (commercially known as the PartoSure test) has been reported to be the single best predictor of imminent spontaneous delivery within 7 days of a patient presenting with signs, symptoms, or complaints of preterm labor.

Fetal fibronectin (fFN) is a fibronectin protein produced by fetal cells. It is found at the interface of the chorion and the decidua (between the fetal sac and the uterine lining). It has become an important biomarker—the presence of this glycoprotein in the cervical or vaginal secretions indicates that the border between the chorion and deciduas has been disrupted. A positive test indicates an increased risk of preterm birth, and a negative test has a high predictive value. It has been shown that only 1% of women in questionable cases of preterm labor delivered within the next week when the test was negative.

Obstetric ultrasound has become useful in the assessment of the cervix to identify in women at risk for premature delivery or to assess women with increased risk of PTB. A short cervix preterm is undesirable: A cervical length of less than 25 mm at or before 24 weeks of gestational age is the most common definition of cervical incompetence. Cervical weakness, also called cervical incompetence or cervical insufficiency, is a medical condition of pregnancy in which the cervix begins to dilate (widen) and efface (thin) before the pregnancy has reached term. Cervical weakness may cause miscarriage or preterm birth during the second and third trimesters. Short cervical length has been shown to be a marker of preterm birth rather than of cervical weakness.

The treatment options for PTB, in particular preventive treatment options, for typically available outside a hospital or in a hospital are known to a skilled person, and are by way of example also disclosed herein.

Smoking bans are effective in decreasing preterm births. Adoption of specific professional policies can immediately reduce risk of preterm birth. Preventive measures include protection of (pregnant) women from hazardous or night-shift work and to provide them with time for prenatal visits and paid pregnancy-leave. Working over 42 hours per week should be avoided, as well as prolonged standing (over 6 hours per day). Also, night work has been linked to preterm birth. Preconceptional intake of folic acid is recommended to reduce birth defects.

Self-care methods to reduce the risk of preterm birth include proper nutrition, avoiding stress, seeking appropriate medical care, avoiding infections, and the control of preterm birth risk factors (e.g. working long hours while standing on feet, carbon monoxide exposure, domestic abuse, and other factors). Healthy eating can be instituted at any stage of the pregnancy including nutritional adjustments and consuming suggested vitamin supplements. Calcium supplementation in women who have low dietary calcium may reduce the number of negative outcomes including preterm birth.

Smoking cessation reduces the risk or PTB. The use of personal at home uterine monitoring devices to detect contractions and possible preterm births in women at higher risk of having a preterm baby can be a useful treatment, preventive or monitoring measure. These home monitors may not reduce the number of preterm births, however, using these devices may increase the number of unplanned antenatal visits and may reduce the number of babies admitted to special care when compared with women receiving normal antenatal care.

Routine ultrasound examination of the length of the cervix may identify women at risk of preterm labour and tentative evidence suggests ultrasound measurement of the length of the cervix in those with preterm labor can help adjust management and results in the extension of pregnancy.

Reduction of existing risks is another measure to be taken for subjects that have been identified as having an increased risk of PTB.

A number of agents have been suggested to reduce PTB, including low-dose aspirin, fish oil, vitamin C and E, and calcium.

Reduction in activity by the mother—pelvic rest, limited work, bed rest—may be recommended. Increasing medical care by more frequent visits and more education may also be recommended. Use of nutritional supplements such as omega-3 polyunsaturated fatty acids is based on the observation that populations who have a high intake of such agents are at low risk for preterm birth, presumably as these agents inhibit production of proinflammatory cytokines.

Progestogens—often given in the form of vaginal progesterone or hydroxyprogesterone caproate—relax the uterine musculature, maintain cervical length, and possess anti-inflammatory properties; all of which invoke physiological and anatomical changes considered to be beneficial in reducing preterm birth. Progestogen supplementation also reduces the frequency of preterm birth in pregnancies where there is a short cervix.

In preparation for childbirth, the woman's cervix shortens. Preterm cervical shortening is linked to preterm birth and can be detected by ultrasonography. Cervical cerclage is a surgical intervention that places a suture around the cervix to prevent its shortening and widening. Women at risk of PTB can be monitored during pregnancy by sonography, and when shortening of the cervix is observed, the cerclage can be performed.

Tertiary interventions are aimed at women who are about to go into preterm labor, or rupture the membranes or bleed preterm. The use of the fibronectin test and ultrasonography improves the diagnostic accuracy and reduces false-positive diagnosis. While treatments to arrest early labor where there is progressive cervical dilatation and effacement will not be effective to gain sufficient time to allow the fetus to grow and mature further, it may defer delivery sufficiently to allow the mother to be brought to a specialized center that is equipped and staffed to handle preterm deliveries. In a hospital setting women are hydrated via intravenous infusion (as dehydration can lead to premature uterine contractions).

As used herein, “diagnosis” in the context of the present invention relates to the recognition and (early) detection of a clinical condition. Also the assessment of the severity may be encompassed by the term “diagnosis”.

“Prognosis” relates to the prediction of an outcome or a specific risk for a subject. This may also include an estimation of the chance of recovery or the chance of an adverse outcome for said subject.

The methods of the invention may also be used for monitoring, therapy monitoring, therapy guidance and/or therapy control. “Monitoring” relates to keeping track of a patient and potentially occurring complications, e.g. to analyze the progression of the healing process or the influence of a particular treatment or therapy on the health state of the patient.

The term “therapy monitoring” or “therapy control” in the context of the present invention refers to the monitoring and/or adjustment of a therapeutic treatment of said patient, for example by obtaining feedback on the efficacy of the therapy. As used herein, the term “therapy guidance” refers to application of certain therapies, therapeutic actions or medical interventions based on the value/level of one or more biomarkers and/or clinical parameter and/or clinical scores. This includes the adjustment of a therapy or the discontinuation of a therapy.

In the present invention, the terms “risk assessment” and “risk stratification” relate to the grouping of subjects into different risk groups according to their further prognosis. Risk assessment also relates to stratification for applying preventive and/or therapeutic measures. The term “therapy stratification” in particular relates to grouping or classifying patients into different groups, such as risk groups or therapy groups that receive certain differential therapeutic measures depending on their classification. The term “therapy stratification” also relates to grouping or classifying patients with infections or having symptoms of an infectious disease into a group that are not in need to receive certain therapeutic measures.

In embodiments of the invention, the pregnant subject is under remote patient management or will be admitted to remote patient management as a result of the outcome of the method of the present invention. For example, if a risk of PTB is determined in a patient by means of the method of the invention, the pregnant subject may be admitted for remote management. As used herein the term “remote patient management” preferably refers to a preventive or therapeutic approach for remotely managing pregnant subjects, in particular pregnant subjects with a risk of PTB, in which data on the health status of a subject is repeatedly collected at the site of the subject (i.e. out-patients) and transmitted to remote (geographically separated) medical personnel or an automated system, which may or may not act upon said data to contact the patient, give advice to the subject, initiate or change concomitant treatments or take any other medical intervention for ameliorating and/or stabilizing the health state of the patient.

A remote patient management preferably encompasses a telemonitoring on health status of the subject as well as telemedical interventions, guideline-based ambulatory care and/or structured patient education.

Telemonitoring preferably refers to the repeated data collection at the site of the subject and its remote transmission to a monitoring system or device allowing for review by medical personnel or an automated medical system.

As used herein the term “remote patient management” preferably refers to a therapeutic approach for remotely managing patients with a infectious disease, in which data on the health status of a patient is repeatedly collected at the site of the patient (i.e. out-patients) and transmitted to remote (geographically separated) medical personnel or an automated system, which may or may not act upon said data to contact the patient, give advice to the patient, initiate or change concomitant treatments or take any other medical intervention for ameliorating and/or stabilizing the health state of the patient.

A remote patient management preferably encompasses a telemonitoring on health status of the patient as well as telemedical interventions, guideline-based ambulatory care and/or structured patient education.

Telemonitoring preferably refers to the repeated data collection at the site of the subject and its remote transmission to a monitoring system or device allowing for review by medical personnel or an automated medical system.

It is understood that in the context of the present invention “determining the level of a biomarker or fragment(s) thereof” or the like refers to any means of determining a respective biomarker, such as in particular MMP9 and/or PAPP-A2, or a fragment thereof. The fragment can have any length, e.g. at least about 5, 10, 20, 30, 40, 50 or 100 amino acids, so long as the fragment allows the unambiguous determination of the level of the respective biomarker or fragment thereof.

Matrix metallopeptidase 9 (MMP-9) (Human: UniProt P14780; Ensembl ENSG00000100985; Entrez Gene ID 4318), also known as 92 kDa type IV collagenase, 92 kDa gelatinase or gelatinase B (GELB), is a matrixin, a class of enzymes that belong to the zinc-metalloproteinases family involved in the degradation of the extracellular matrix. In humans the MMP9 gene encodes for a signal peptide, a propeptide, a catalytic domain with inserted three repeats of fibronectin type II domain followed by a C-terminal hemopexin-like domain. Proteins of the matrix metalloproteinase (MMP) family are involved in the breakdown of extracellular matrix in normal physiological processes, such as embryonic development, reproduction, angiogenesis, bone development, wound healing, cell migration, learning and memory, as well as in pathological processes, such as arthritis, intracerebral hemorrhage, and metastasis. Most MMPs are secreted as inactive proproteins which are activated when cleaved by extracellular proteinases. The enzyme encoded by this gene degrades type IV and V collagens and other extracellular matrix proteins. Studies in rhesus monkeys suggest that the enzyme is involved in IL-8-induced mobilization of hematopoietic progenitor cells from bone marrow, and murine studies suggest a role in tumor-associated tissue remodeling. MMP9 plays several important functions within neutrophil action, such as degrading extracellular matrix, activation of IL-1β, and cleavage of several chemokines. In a mouse model, MMP9 deficiency resulted in resistance to endotoxin shock, suggesting that MMP9 is important in sepsis. MMP9 may play an important role in angiogenesis and neovascularization. MMP9 has been found to be associated with numerous pathological processes, including cancer, placental malaria, immunologic and cardiovascular diseases. For example, elevated MMP9 levels can be found in the cases of rheumatoid arthritis and focal brain ischemia.

MMP9 is synthesized as preproenzyme of 707 amino-acid residues, including a 19 amino acid signal peptide and secreted as an inactive pro-MMP. The human MMP9 proenzyme consists of five domains. The amino-terminal propeptide, the zinc-binding catalytic domain and the carboxyl-terminal hemopexin-like domain are conserved. Its primary structure comprises several domain motifs. The propeptide domain is characterized by a conserved PRCGVPD sequence. The Cys within this sequence is known as the “cysteine switch”. It ligates the catalytic zinc to maintain the enzyme in an inactive state. Activation is achieved through an interacting protease cascade involving plasmin and stromelysin 1 (MMP-3). Plasmin generates active MMP-3 from its zymogen. Active MMP-3 cleaves the propeptide from the 92-kDa pro-MMP-9, yielding an 82-kDa enzymatically active enzyme. In the active enzyme a substrate, or a fluorogenic activity probe, replaces the propetide in the enzyme active site where it is cleaved. The catalytic domain contains two zinc and three calcium atoms. The catalytic zinc is coordinated by three histidines from the conserved HEXXHXXGXXH binding motif. The other zinc atom and the three calcium atoms are structural. A conserved methionine, which forms a unique “Met-turn” structure categorizes MMP9 as a metzincin. Three type II fibronectin repeats are inserted in the catalytic domain, although these domains are omitted in most crystallographic structures of MMP9 in complex with inhibitors. The active form of MMP9 also contains a C-terminal hemopexin-like domain. This domain is ellipsoidal in shape, formed by four β-propeller blades and an α-helix. Each blade consists of four antiparallel β-strands arranged around a funnel-like tunnel that contains two calcium and two chloride ions. The hemopexin domain is important to facilitate the cleavage of triple helical interstitial collagens.

Tu et al. (Obstet. Gynecol. 1998) reported that MMP-9 levels remain unchanged throughout pregnancy until the onset of spontaneous labor when there is a three-fold increase. They concluded that MMP-9 levels obtained prior to presentation for delivery do not appear to predict spontaneous preterm birth (accross pregnancy). However, as disclosed herein, it was surprisingly found that MMP-9 allows to predict spontaneous preterm birth several weeks or months before the onset.

Tao et al. (Med. Scoi. Monit., 2019; 25:4513-4520) reported that increase of cytokines (including MMP9) is a predictor of labor for patient at term (38-39 weeks of gestation) and not for preterm birth which by definition occurs before 37 weeks of gestation. Surprisingly, the results reported herein demonstrate that MMP9 concentration change occurs several months before delivery and labor onset.

Pappalysin-2 (PAPP-A2; pregnancy-associated plasma protein-A2) is a protein that in humans is encoded by the PAPPA2 gene (Human: UniProt Q9BXP8; Ensembl ENSG00000116183; Entrez Gene ID 60676).

Pregnancy-associated plasma protein-A (PAPP-A; Human: UniProt Q13219; Ensembl ENSG00000182752; Entrez Gene ID 5069) and its paralog PAPP-A2 are the two best-characterized IGFBP-cleaving enzymes. The two enzymes (often referred to as pappalysins) regulate the liberation of IGF-I in a highly controlled manner. Pregnancy-associated plasma protein-A (PAPP-A) and PAPP-A2 comprise the only two known members of the pappalysin family of metalloproteinases, sharing 45% amino acid identity. They are responsible for proteolytic cleavage of a subset of IGF-binding proteins (IGFBPs), through which they increase IGF availability and potentiate its growth stimulatory effects. PAPP-A has been suggested as an accomplice in several types of cancer and has been extensively studied due to its biomarker potential. Although PAPP-A2 was recently established as a regulator of the IGF axis in human physiology, the biology of PAPP-A2 is poorly understood compared to PAPP-A, and there are currently no reports linking PAPP-A2 protein and cancer mortality.

PAPP-A specifically cleaves IGFBP-2, -4, and -5 and is widely expressed in multiple tissues, including those of tumor origin, where it tethers to cell surfaces. Thus, PAPP-A causes a release of bioactive IGF in close proximity to the IGF-IR. Shifts in PAPP-A levels have been suggested to modify the relationship between bound and free IGF in various neoplasms. In patients with lung cancer, serum PAPP-A levels have been shown to be elevated, and down-regulation of PAPP-A expression decreases lung cancer progression in vivo. In ovarian cancer, in ascites, which surrounds the ovarian tumor in the abdominal cavity and is a negative prognostic factor, PAPP-A levels were 46-fold higher as compared to serum from the same patient.

Similar to PAPP-A, placentally derived PAPP-A2 is abundantly present in the circulation throughout pregnancy, but the protein is also detectable in non-pregnant men and women. However, PAPP-A2 has generally not been investigated in human pathologic conditions outside pregnancy. PAPP-A2 exhibits proteolytic activity against IGFBP-3 and -5, but unlike PAPP-A, PAPP-A2 does not show surface tethering. PAPP-A2 deficiency cases, present with short stature and severe perturbations in the IGF system.

The determination of various forms of the biomarkers that can be determined in the method of the invention, such as in particular MMP9 and/or PAPP-A2, and fragments thereof also encompass measuring and/or detecting specific sub-regions of the biomarkers, for example by employing antibodies or other affinity reagents directed against a particular portion of the molecules, or by determining the presence and/or quantity of the molecules by measuring a portion of the protein using mass spectrometry.

The methods and kits of the present invention can also comprise determining at least one further biomarker, marker, clinical score and/or parameter in addition to MMP9 and/or PAPP-A2.

Lactoferrin (Human: UniProt P02788; Ensembl ENSG00000012223; Entrez Gene ID 4057), also known as lactotransferrin (LTF), is a multifunctional protein of the transferrin family. Lactoferrin is a globular glycoprotein with a molecular mass of about 80 kDa that is widely represented in various secretory fluids, such as milk, saliva, tears, and nasal secretions. Lactoferrin is also present in secondary granules of PMNs and is secreted by some acinar cells. Lactoferrin can be purified from milk or produced recombinantly. Human colostrum (“first milk”) has the highest concentration, followed by human milk, then cow milk (150 mg/L). Lactoferrin levels in bodily samples and fluids are being used as a biomarkers. For example, LTF in tear fluid have been shown to decrease in dry eye diseases such as Sjögren's syndrome. A rapid, portable test utilizing microfluidic technology has been developed to enable measurement of lactoferrin levels in human tear fluid at the point-of-care with the aim of improving diagnosis of Sjögren's syndrome and other forms of dry eye disease.

Chromogranin A (CGA; Human: UniProt P10645; Ensembl ENSG00000276781, ENSG00000100604; Entrez Gene ID 1113), is is a member of the granin family of neuroendocrine secretory proteins. As such, it is located in secretory vesicles of neurons and endocrine cells such as islet beta cell secretory granules in the pancreas. In humans, chromogranin A protein is encoded by the CHGA gene. Chromogranin A is the precursor to several functional peptides including vasostatin-1, vasostatin-2, pancreastatin, catestatin and parastatin. These peptides negatively modulate the neuroendocrine function of the releasing cell (autocrine) or nearby cells (paracrine). Chromogranin A induces and promotes the generation of secretory granules such as those containing insulin in pancreatic islet beta cells. Chromogranin A is elevated in pheochromocytomas. It has been identified as autoantigen in type 1 diabetes. It is used as an indicator for pancreas and prostate cancer and in carcinoid syndrome. It might play a role in early neoplasic progression. Chromogranin A is cleaved by an endogenous prohormone convertase to produce several peptide fragments.

INHA is a heterodimer of inhibin, beta A (also known as INHBA, UniProt P08476), and inhibin, alpha (also known as INHA, UniProt P05111). The inhibin alpha subunit joins either the beta A or beta B subunit to form a pituitary FSH secretion inhibitor. Inhibin has been shown to regulate gonadal stromal cell proliferation negatively and to have tumour-suppressor activity. In addition, serum levels of inhibin have been shown to reflect the size of granulosa-cell tumors and can therefore be used as a marker for primary as well as recurrent disease. However, in prostate cancer, expression of the inhibin alpha-subunit gene was suppressed and was not detectable in poorly differentiated tumor cells. Furthermore, because expression in gonadal and various extragonadal tissues may vary severalfold in a tissue-specific fashion, it is proposed that inhibin may be both a growth/differentiation factor and a hormone.

ACTA, also called Activin A, is a homodimer of the inhibin, beta A chain (UniProt P08476). Activin and inhibin are two closely related protein complexes that have almost directly opposite biological effects. The activin and inhibin protein complexes are both dimeric in structure, and, in each complex, the two monomers are linked to one another by a single disulfide bond.

Palmitoleoyl-protein carboxylesterase is also called NOTUM is a protein (UniProt Q6P988) that in humans is encoded by the NOTUM gene. It acts as a key negative regulator of the Wnt signaling pathway by specifically mediating depalmitoleoylation of WNT proteins. NOTUM expression was found to be increased in metastatic cells. Proliferation was suppressed by inhibiting expression of NOTUM. Knockdown of NOTUM genes inhibited proliferation as well as migration, with possible involvement of p38 and c-JUN N-terminal kinase in this process. Furthermore, it was shown that notum and glypican-1 and glypican-3 gene expression during colorectal cancer (CRC) development. Kinetic and mass spectrometric analyses of human proteins show that Notum is a carboxylesterase that removes an essential palmitoleate moiety from Wnt proteins and thus constitutes the first known extracellular protein deacylase. Overexpression of NOTUM is associated with hepatocellular carcinoma.

Pregnancy Specific Beta-1-Glycoprotein 3 (PSG3; NCBI Entrez Gene ID: 5671; Ensembl: ENSG00000221826, UniProt: Q16557) is associated with Twin-To-Twin Transfusion Syndrome and Apnea, Obstructive Sleep. Among its related pathways are Cell surface interactions at the vascular wall and Response to elevated platelet cytosolic Ca2+. The human pregnancy-specific glycoproteins (PSGs) are a family of proteins that are synthesized in large amounts by placental trophoblasts and released into the maternal circulation during pregnancy.

Macrophage Stimulating 1 Like (MST1L) (Entrez Gene: 11223; Ensembl: ENSG00000186715; UniProt: Q2TV78) is considered to be a pro-inflammtory factor.

Sex hormone-binding globulin (SHBG) or sex steroid-binding globulin (SSBG) is a glycoprotein that binds to androgens and estrogens (UniProt 004278; Entrez 6462). SHBG is produced mostly by the liver and is released into the bloodstream. Other sites that produce SHBG include the brain, uterus, testes, and placenta.[12] Testes-produced SHBG is called androgen-binding protein. Reference ranges for blood tests for SHBG have been developed, Adult female, premenopausal: 40-120 nmol/L; adult female, postmenopausal: 28-112 nmol/L; Adult male: 20-60 nmol/L; Infant (1-23 months): 60-252 nmol/L; Prepubertal (2 years-8 years): 72-220 nmol/L; Pubertal female: 36-125 nmol/L; pubertal male: 16-100 nmol/L. SHBG levels are decreased by androgens, administration of anabolic steroids, polycystic ovary syndrome, hypothyroidism, obesity, Cushing's syndrome, and acromegaly. Low SHBG levels increase the probability of Type 2 Diabetes. SHBG levels increase with estrogenic states (oral contraceptives), pregnancy, hyperthyroidism, cirrhosis, anorexia nervosa, and certain drugs. Long-term calorie restriction of more than 50 percent (in rodents) increases SHBG, while lowering free and total testosterone and estradiol. DHEA-S, which lacks affinity for SHBG, is not affected by calorie restriction. Polycystic Ovarian Syndrome is associated with insulin resistance and excess insulin lowers SHBG, which increases free testosterone levels.

Soluble fms-like tyrosine kinase-1 (sFlt-1 or sVEGFR-1; UniProt P17948) is a tyrosine kinase protein with antiangiogenic properties. A non-membrane associated splice variant of VEGF receptor 1 (Flt-1), sFlt-1 binds the angiogenic factors VEGF (vascular endothelial growth factor) and PIGF (placental growth factor), reducing blood vessel growth through reduction of free VEGF and PIGF concentrations. In humans, sFlt-1 is important in the regulation of blood vessel formation in diverse tissues, including the kidneys, cornea, and uterus. Abnormally high levels of sFIt-1 have been implicated in the pathogenesis of preeclampsia. PIGF and sFlt-1 concentrations measured by immunoassay in maternal blood improve the prognostic possibilities in preeclampsia, which is typically diagnosed solely on the basis of clinical symptoms, proteinuria, and uterine artery Doppler velocimetry. Notably, increases in sFlt-1 and decreases in PIGF and VEGF can be detected at least five weeks before the onset of preeclamptic symptoms, potentially facilitating earlier diagnosis and treatment. sFlt-1 changes are most predictive of early-onset preeclampsia; cases of preeclampsia incident late in pregnancy typically are accompanied only by small decreases in PIGF. However, sFlt-1 elevation is also associated with other obstetric conditions such as non-preeclampsia interuterine growth retardation of the fetus, limiting its use as a discriminatory biomarker for preeclampsia. Additionally, sensitivity and specificity of sFlt-1 testing is generally considered too low to enable it to serve as an effective predictor of preeclampsia.

Disintegrin and metalloproteinase domain-containing protein 12 (previously Meltrin; also called ADAM12 or ADA12; UniProt Q43184; Entrez 8038) is an enzyme that in humans is encoded by the ADAM12 gene. ADAM12 has two splice variants: ADAM12-L, the long form, has a transmembrane region and ADAM12-S, a shorter variant, is soluble and lacks the transmembrane and cytoplasmic domains. ADAM 12, a metalloprotease that binds insulin growth factor binding protein-3 (IGFBP-3), appears to be an effective early Down syndrome marker. Decreased levels of ADAM 12 may be detected in cases of trisomy 21 as early as 8 to 10 weeks gestation. Maternal serum ADAM 12 and PAPP-A levels at 8 to 9 weeks gestation in combination with maternal age yielded a 91% detection rate for Down syndrome at a 5% false-positive rate. When nuchal translucency data from approximately 12 weeks gestation was added, this increased the detection rate to 97%. ADAM12 has also been implicated in the development of pathology in various cancers, hypertension, liver fibrogenesis, and asthma. In asthma, ADAM12 is upregulated in lung epithelium in response to TNF-alpha.

Ficolin-3 is a protein (also called FCN3, Uniprot 075636; Entrez 8547) that in humans is encoded by the FCN3 gene. Ficolin-3 was initially identified as H-ficolin, in which H is after the Hakata antigen that was previously found as an autoantigen in patients who lived in the city of Hakata. Ficolins are a group of proteins which consist of a collagen-like domain and a fibrinogen-like domain. In human serum, there are two types of ficolins, both of which have lectin activity. The protein encoded by this gene is a thermolabile beta-2-macroglycoprotein found in all human serum and is a member of the ficolin/opsonin p35 lectin family. The protein, which was initially identified based on its reactivity with sera from patients with systemic lupus erythematosus, has been shown to have a calcium-independent lectin activity. The protein can activate the complement pathway in association with MASPs and sMAP, thereby aiding in host defense through the activation of the lectin pathway. Alternative splicing occurs at this locus and two variants, each encoding a distinct isoform, have been identified.

Placental growth factor (PGF or PIGF, UniProt P49763; Entrez 5228) is a member of the VEGF (vascular endothelial growth factor) sub-family—a key molecule in angiogenesis and vasculogenesis, in particular during embryogenesis. The main source of PIGF during pregnancy is the placental trophoblast. PIGF is also expressed in many other tissues, including the villous trophoblast. The placental growth factor gene is a protein-coding gene and a member of the vascular endothelial growth factor (VEGF) family. The PIGF gene is expressed only in human umbilical vein endothelial cells (HUVE) and the placenta. PIGF is ultimately associated with angiogenesis. Specifically, PIGF plays a role in trophoblast growth and differentiation. Trophoblast cells, specifically extravillous trophoblast cells, are responsible for invading the uterine wall and the maternal spiral arteries. The extravillous trophoblast cells produce a blood vessel of larger diameter for the developing fetus that is independent of maternal vasoconstriction. This is essential for increased blood flow and reduced resistance. Proper development of blood vessels in the placenta is crucial for the higher blood requirement of the fetus later in pregnancy. Under normal physiologic conditions, PGF is also expressed at a low level in other organs including the heart, lung, thyroid, and skeletal muscle.

Serum levels of PIGF and sFlt-1 (soluble fms-like tyrosine kinase-1, also known as soluble VEGF receptor-1) are altered in women with preeclampsia. Studies show that in both early and late onset preeclampsia, maternal serum levels of sFlt-1 are higher and PIGF lower in women presenting with preeclampsia. In addition, placental sFlt-1 levels were significantly increased and PIGF decreased in women with preeclampsia as compared to those with uncomplicated pregnancies. This suggests that placental concentrations of sFIt-1 and PIGF mirror the maternal serum changes. This is consistent with the view that the placenta is the main source of sFlt-1 and PIGF during pregnancy. PIGF is a potential biomarker for preeclampsia, a condition in which blood vessels in the placenta are too narrow, resulting in high blood pressure. As mentioned before, extravillous trophoblast cells invade maternal arteries. Improper differentiation may result in hypo-invasion of these arteries and thus failure to widen enough. Studies have found low levels of PIGF in women who were diagnosed with preeclampsia later in their pregnancy.

Preferred peptide sequence of biomarkers to be determined in the context of the method of the invention are disclosed in the examples below. Such peptide sequence may be useful for MS determination of the markers.

In embodiments, the invention comprises determining and/or assessing clinical parameter of the pregnant subject, such as clinical history, weight, body mass index (BMI), result of cervical examination (e.g. cervical length or cervical shape), result of ultra sound or other clinical imaging method, age of the pregnant subject, FMF algorithm, smoking habits, blood pressure. As used herein, a parameter is a characteristic, feature, or measurable factor that can help in defining a particular system and can be used for improving determining the method of the present invention. A parameter is an important element for health- and physiology-related assessments, such as a disease/disorder/clinical condition risk, preferably organ dysfunction(s). Furthermore, a parameter is defined as a characteristic that is objectively measured and evaluated as an indicator of normal biological processes, pathogenic processes, or pharmacologic responses to a therapeutic intervention. Exemplary parameters that can be used in the context of the invention can be selected from the group consisting body mass index (BMI), body weight, age, sex, IGS II, liquid intake, white blood cell count, sodium, potassium, temperature, blood pressure, dopamine, bilirubin, respiratory rate, partial pressure of oxygen, family history, ethnicity, cystoscopy report, heart rate, antihypertensive treatment, lymphocyte count, body temperature, presence of diabetes mellitus, blood glucose levels, (current) smoking habits, and imaging methods as such as ultrasound examination, CT scan, PET imaging or X-ray. In the context of the present invention, particularly useful parameter may be clinical history, weight, body mass index (BMI), result of cervical examination (e.g. cervical length or cervical shape), result of ultra sound or other clinical imaging method, age of the pregnant subject, FMF algorithm, smoking habits, blood pressure. Such parameters may additionally be assessed in combination with the methods described herein in order to improve assay implementation and diagnostic statements.

The FMF (Fetal Medicine Foundation) algorithm is a specific diagnostic method (i.e. algorithm) for the risk assessment of preeclampsia. Risks can be derived from maternal history and any combinations of biomarkers. Useful markers at 11-14 weeks are mean arterial pressure (MAP), uterine artery PI (UTPI) and serum PIGF (or PAPP-A when PIGF is not available). The values for PIGF and PAPP-A depend on maternal characteristics and reagents used for analysis and they therefore need to be converted into MoMs.

The present invention has the following advantages over the conventional methods: the inventive methods and the kits are fast, objective, easy to use and precise. The methods and kits of the invention relate to biomarkers and optionally clinical parameters that are easily measurable in routine methods, because the levels of MMP9 and/or PAPP-A2 and the other potentially assessed biomarkers can be determined in routinely obtained samples, such as in particular in blood samples (including venous and capillary blood, serum and plasma samples), or further biological fluids or samples obtained from a subject.

As used herein, terms such as “biomarker”, marker”, “surrogate”, “prognostic marker”, “factor” or or “biological marker” are used interchangeably and relate to measurable and quantifiable biological markers (e.g., specific protein or enzyme concentration or a fragment thereof, specific hormone concentration or a fragment thereof, or presence of biological substances or a fragment thereof) which serve as indices for health- and physiology-related assessments, such as a disease/disorder/clinical condition risk, preferably an adverse event. A marker or biomarker is defined as a characteristic that can be objectively measured and evaluated as an indicator of normal biological processes, pathogenic processes, or pharmacologic responses to a therapeutic intervention. Biomarkers may be measured in a sample (as a blood, plasma, urine, or tissue test).

Further biomarkers and/or parameters to be used and determined in the context of the present invention can be selected from the group consisting of a level of lactate in said sample, a level of procalcitonin (PCT) in said sample, the sequential organ failure assessment score (SOFA score) of said subject, optionally the quick SOFA score, the simplified acute physiology score (SAPSII) of said subject, the Acute Physiology and Chronic Health Evaluation II (APACHE II) score of said subject and a level of the soluble fms-like tyrosine kinase-1 (sFlt-1), Histone H2A, Histone H2B, Histone H3, Histone H4, calcitonin, Endothelin-1 (ET-1), Arginine Vasopressin (AVP), Atrial Natriuretic Peptide (ANP), Neutrophil Gelatinase-Associated Lipocalin (NGAL), Troponin, Brain Natriuretic Peptide (BNP), C-Reactive Protein (CRP), Pancreatic Stone Protein (PSP), Triggering Receptor Expressed on Myeloid Cells 1 (TREM1), Interleukin-6 (IL-6), Interleukin-1, Interleukin-24 (IL-24), Interleukin-22 (IL-22), Interleukin (IL-20) other ILs, Presepsin (sCD14-ST), Lipopolysaccharide Binding Protein (LBP), Alpha-1-Antitrypsin, Matrix Metalloproteinase 2 (MMP2), Metalloproteinase 2 (MMP8), Matrix Metalloproteinase 9 (MMP9), Matrix Metalloproteinase 7 (MMP7, Placental growth factor (PIGF), Chromogranin A, S100A protein, S100B protein and Tumor Necrosis Factor α (TNFα), Neopterin, Alpha-1-Antitrypsin, pro-arginine vasopressin (AVP, proAVP or Copeptin), procalcitonin, atrial natriuretic peptide (ANP, pro-ANP), Endothelin-1, CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, FAM19A1, FAM19A2, FAM19A3, FAM19A4, FAM19A5, CLCF1, CNTF, IL11, IL31, IL6, Leptin, LIF, OSM, IFNA1, IFNA10, IFNA13, IFNA14, IFNA2, IFNA4, IFNA7, IFNB1, IFNE, IFNG, IFNZ, IFNA8, IFNA5/IFNaG, IFNω/IFNW1, BAFF, 4-1BBL, TNFSF8, CD40LG, CD70, CD95L/CD178, EDA-A1, TNFSF14, LTA/TNFB, LTB, TNFa, TNFSF10, TNFSF11, TNFSF12, TNFSF13, TNFSF15, TNFSF4, TRAIL, IP-10, IL18, IL18BP, IL1A, IL1B, IL1F10, IL1F3/IL1RA, IL1F5, IL1F6, IL1F7, IL1F8, IL1RL2, IL1F9, IL33 or a fragment thereof. Further markers comprise membrane microparticle, platelet count, mean platelet volume (MPV), sCD14-ST, prothrombinase, antithrombin and/antithrombin activity, cationic protein 18 (CAP18), von Willebrand factor (vWF)-cleaving proteases, lipoproteins in combination with CRP, fibrinogen, fibrin, B2GP1, GPIIb-IIIa, non-denatured D-dimer of fibrin, platelet factor 4, histones and a PT-Assay.

In embodiments, the invention comprises determing a level of PCT in a sample of the pregnant subject. As used herein, “procalcitonin” or “PCT” relates to a peptide spanning amino acid residues 1-116, 2-116, 3-116, or fragments thereof, of the procalcitonin peptide. PCT is a peptide precursor of the hormone calcitonin. Thus the length of procalcitonin fragments is at least 12 amino acids, preferably more than 50 amino acids, more preferably more than 110 amino acids. PCT may comprise post-translational modifications such as glycosylation, liposidation or derivatization. Procalcitonin is a precursor of calcitonin and katacalcin. Thus, under normal conditions the PCT levels in the circulation are very low (<about 0.05 ng/ml).

The level of PCT in the sample of the subject can be determined by immunoassays as described herein. As used herein, the level of ribonucleic acid or deoxyribonucleic acids encoding “procalcitonin” or “PCT” can also be determined. Methods for the determination of PCT are known to a skilled person, for example by using products obtained from Thermo Fisher Scientific.

In embodiments, the invention comprises determing a level of ADM, preferably proADM, more preferably MR-proADM, in a sample of the pregnant subject. The peptide adrenomedullin (ADM) was discovered as a hypotensive peptide comprising 52 amino acids, which had been isolated from a human phenochromocytome (Kitamura et al., 1993). Adrenomedullin (ADM) is encoded as a precursor peptide comprising 185 amino acids (“preproadrenomedullin” or “pre proADM”).

ADM comprises the positions 95-146 of the pre-proADM amino acid sequence and is a splice product thereof. “Proadrenomedullin” (“proADM”) refers to pre-proADM without the signal sequence (amino acids 1 to 21), i.e. to amino acid residues 22 to 185 of pre-proADM. “Midregional proadrenomedullin” (“MR-proADM”) refers to the amino acids 42 to 95 of pre-proADM. It is also envisaged herein that a peptide and fragment thereof of pre-proADM or MR-proADM can be used for the herein described methods. For example, the peptide or the fragment thereof can comprise the amino acids 22-41 of pre-proADM (PAMP peptide) or amino acids 95-146 of pre-proADM (mature adrenomedullin, including the biologically active form, also known as bio-ADM). A C-terminal fragment of proADM (amino acids 153 to 185 of pre proADM) is called adrenotensin. Fragments of the proADM peptides or fragments of the MR-proADM can comprise, for example, at least about 5, 10, 20, 30 or more amino acids. Accordingly, the fragment of proADM may, for example, be selected from the group consisting of MR-proADM, PAMP, adrenotensin and mature adrenomedullin, preferably herein the fragment is MR-proADM.

As used herein, the term “sample” is a biological sample that is obtained or isolated from the patient or subject. “Sample” as used herein may, e.g., refer to a sample of bodily fluid or tissue obtained for the purpose of analysis, diagnosis, prognosis, or evaluation of a subject of interest, such as a patient. Preferably herein, the sample is a sample of a bodily fluid, such as blood, serum, plasma, cerebrospinal fluid, urine, saliva, sputum, pleural effusions, cells, a cellular extract, a tissue sample, any tissue sample from the upper or lower respiratory tract, a tissue biopsy, a stool sample and the like. Particularly, the sample is blood, blood plasma, blood serum.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood), for example for at least 15 minutes at 2000 to 3000 g.

“Serum” in the context of the present invention is the liquid fraction of whole blood that is collected after the blood is allowed to clot. When coagulated blood (clotted blood) is centrifuged serum can be obtained as supernatant.

As used herein, “urine” is a liquid product of the body secreted by the kidneys through a process called urination (or micturition) and excreted through the urethra.

In the context of the present invention, the term “medical treatment” or “treatment” comprises various treatments and therapeutic strategies, which comprise, without limitation.

A skilled person is capable of determining which of the treatments described herein require administration in a hospital setting.

A skilled person is also capable of determining which medical conditions, and which degrees of severity of such medical conditions, require treatments only (or primarily) available in hospital settings.

According to the present invention MMP9 and/or PAPP-A2 and/or other markers and/or clinical parameters are employed as markers for the diagnosis, prognosis, prediction, risk assessment and/or risk stratification of PTB in a pregnant subject.

A skilled person is capable of obtaining or developing means for the identification, measurement, determination and/or quantification of any one of the above biomarkers, or fragments or variants thereof, as well as the other markers of the present invention according to standard molecular biological practice.

The level of MMP9 and/or PAPP-A2 or fragments thereof as well as the levels of other markers of the present invention can be determined by any assay that reliably determines the concentration or relative abundance of the marker. Particularly, mass spectrometry (MS) and/or immunoassays can be employed as exemplified in the appended examples. As used herein, an immunoassay is a biochemical test that measures the presence or concentration of a macromolecule/polypeptide in a solution through the use of an antibody or antibody binding fragment or immunoglobulin.

Methods of determining the biomarkers of the invention include, without limitation, mass spectrometry (MS), luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats such as for instance immunochromatographic strip tests, rare cryptate assay, and automated systems/analyzers.

Determination of MMP9 and/or PAPP-A2 and optionally other markers based on antibody recognition is a preferred embodiment of the invention. As used herein, the term, “antibody” refers to immunoglobulin molecules and immunologically active portions of immunoglobulin (lg) molecules, i.e., molecules that contain an antigen binding site that specifically binds (immuno reacts with) an antigen. According to the invention, the antibodies may be monoclonal as well as polyclonal antibodies. Particularly, antibodies that are specifically binding to a respective biomarker of the invention are used.

An antibody is considered to be specific, if its affinity towards the molecule of interest is at least 50-fold higher, preferably 100-fold higher, most preferably at least 1000-fold higher than towards other molecules comprised in a sample containing the molecule of interest. It is well known in the art how to develop and to select antibodies with a given specificity. In the context of the invention, monoclonal antibodies are preferred. The antibody or the antibody binding fragment binds specifically to the herein defined markers or fragments thereof. Further, biomarker specific antibodies or an antibody binding fragments can used or provided in the methods and kits of the invention.

Instead of antibodies, other capture molecules or molecular scaffolds that specifically and/or selectively recognize the respective biomarkers of the invention may be encompassed by the scope of the present invention. Herein, the term “capture molecules” or “molecular scaffolds” comprises molecules which may be used to bind target molecules or molecules of interest from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may, for instance, be mediated by ionic, van-der-Waals, pi-pi, sigma-pi, hydrophobic or hydrogen bond interactions or a combination of two or more of the aforementioned interactions or covalent interactions between the capture molecules or molecular scaffold and the target molecules or molecules of interest. In the context of the present invention, capture molecules or molecular scaffolds may for instance be selected from the group consisting of a nucleic acid molecule, a carbohydrate molecule, a PNA molecule, a protein, a peptide and a glycoprotein. Capture molecules or molecular scaffolds include, for example, aptamers, DARpins (Designed Ankyrin Repeat Proteins). Affimers and the like are included.

In certain aspects of the invention, the method is an immunoassay comprising the steps of:
a) contacting the sample with
   i. a first antibody or an antigen-binding fragment or derivative thereof specific for a first epitope of MMP9 and/or PAPP-A2, or fragments thereof, and
   ii. a second antibody or an antigen-binding fragment or derivative thereof specific for a second MMP9 and/or PAPP-A2, or fragments thereof; and
b) detecting the binding of the two antibodies or antigen-binding fragments or derivates thereof to said MMP9 and/or PAPP-A2.

In one embodiment, the method is an immunoassay comprising the steps of:
a) contacting the sample with
   i. a first antibody or an antigen-binding fragment or derivative thereof specific for a first epitope of MMP9 or fragments thereof, and
   ii. a second antibody or an antigen-binding fragment or derivative thereof specific for a second MMP9 or fragments thereof; and
b) detecting the binding of the two antibodies or antigen-binding fragments or derivates thereof to said MMP9.

In one embodiment, the method is an immunoassay comprising the steps of:
a) contacting the sample with
   i. a first antibody or an antigen-binding fragment or derivative thereof specific for a first epitope of PAPP-A2 or fragments thereof, and
   ii. a second antibody or an antigen-binding fragment or derivative thereof specific for a second PAPP-A2 or fragments thereof; and
b) detecting the binding of the two antibodies or antigen-binding fragments or derivates thereof to said PAPP-A2.

Preferably, one of the antibodies can be labeled and the other antibody can be bound to a solid phase or can be bound selectively to a solid phase. In a particularly preferred aspect of the assay, one of the antibodies is labeled while the other is either bound to a solid phase or can be bound selectively to a solid phase. The first antibody and the second antibody can be present dispersed in a liquid reaction mixture, and wherein a first labeling component which is part of a labeling system based on fluorescence or chemiluminescence extinction or amplification is bound to the first antibody, and a second labeling component of said labeling system is bound to the second antibody so that, after binding of both antibodies a measurable signal which permits detection of the resulting sandwich complexes in the measuring solution is generated. The labeling system can comprise a rare earth cryptate or chelate in combination with a fluorescent or chemiluminescent dye, in particular of the cyanine type.

In a preferred embodiment, the method is executed as heterogeneous sandwich immunoassay, wherein one of the antibodies is immobilized on an arbitrarily chosen solid phase, for example, the walls of coated test tubes (e.g. polystyrol test tubes; coated tubes; CT) or microtiter plates, for example composed of polystyrol, or to particles, such as for instance magnetic particles, whereby the other antibody has a group resembling a detectable label or enabling for selective attachment to a label, and which serves the detection of the formed sandwich structures. A temporarily delayed or subsequent immobilization using suitable solid phases is also possible.

The method according to the present invention can furthermore be embodied as a homogeneous method, wherein the sandwich complexes formed by the antibody/antibodies and the marker of the invention, in particular MMP9 and/or PAPP-A2, which is to be detected remains suspended in the liquid phase. In this case it is preferred, that when two antibodies are used, both antibodies are labeled with parts of a detection system, which leads to generation of a signal or triggering of a signal if both antibodies are integrated into a single sandwich. Such techniques are to be embodied in particular as fluorescence enhancing or fluorescence quenching detection methods. A particularly preferred aspect relates to the use of detection reagents which are to be used pair-wise, such as for example the ones which are described in U.S. Pat. No. 4,882,733, EP0180492 or EP0539477 and the prior art cited therein. In this way, measurements in which only reaction products comprising both labeling components in a single immune-complex directly in the reaction mixture are detected, become possible. For example, such technologies are offered under the brand names TRACE™ (Time Resolved Amplified Cryptate Emission) or KRYPTOR™, implementing the teachings of the above-cited applications. Therefore, in particular preferred aspects, a diagnostic device is used to carry out the herein provided method. For example, the level of MMP9 and/or PAPP-A2, or fragments thereof, and/or the level of any further marker of the herein provided method is determined. In particular preferred aspects, the diagnostic device is KRYPTOR™.

The level of the markers of the present invention, e.g. MMP9 or fragments thereof, PAPP-A2 or fragments thereof, or other markers, can also be determined by a mass spectrometric (MS) based methods. Such a method may comprise detecting the presence, amount or concentration of one or more modified or unmodified fragment peptides in said biological sample or a protein digest (e.g. tryptic digest) from said sample, and optionally separating the sample with chromatographic methods, and subjecting the prepared and optionally separated sample to MS analysis. For example, selected reaction monitoring (SRM), multiple reaction monitoring (MRM) or parallel reaction monitoring (PRM) mass spectrometry may be used in the MS analysis, particularly to determine the amounts of proADM or fragments thereof.

Herein, the term “mass spectrometry” or “MS” refers to an analytical technique to identify compounds by their mass. In order to enhance the mass resolving and mass determining capabilities of mass spectrometry, the samples can be processed prior to MS analysis. Accordingly, the invention relates to MS detection methods that can be combined with immuno-enrichment technologies, methods related to sample preparation and/or chromatographic methods, preferably with liquid chromatography (LC), more preferably with high performance liquid chromatography (HPLC) or ultra high performance liquid chromatography (UHPLC). Sample preparation methods comprise techniques for lysis, fractionation, digestion of the sample into peptides, depletion, enrichment, dialysis, desalting, alkylation and/or peptide reduction. However, these steps are optional. The selective detection of analyte ions may be conducted with tandem mass spectrometry (MS/MS). Tandem mass spectrometry is characterized by mass selection step (as used herein, the term “mass selection” denotes isolation of ions having a specified m/z or narrow range of m/z's), followed by fragmentation of the selected ions and mass analysis of the resultant product (fragment) ions.

The skilled person is aware how quantify the level of a marker in the sample by mass spectrometric methods. For example, relative quantification “rSRM” or absolute quantification can be employed as described above.

Moreover, the levels (including reference levels) can be determined by mass spectrometric based methods, such as methods determining the relative quantification or determining the absolute quantification of the protein or fragment thereof of interest.

Relative quantification “rSRM” may be achieved by:

1. Determining increased or decreased presence of the target protein by comparing the SRM (Selected reaction monitoring) signature peak area from a given target fragment peptide detected in the sample to the same SRM signature peak area of the target fragment peptide in at least a second, third, fourth or more biological samples.

2. Determining increased or decreased presence of target protein by comparing the SRM signature peak area from a given target peptide detected in the sample to SRM signature peak areas developed from fragment peptides from other proteins, in other samples derived from different and separate biological sources, where the SRM signature peak area comparison between the two samples for a peptide fragment are normalized for e.g. to amount of protein analyzed in each sample.

3. Determining increased or decreased presence of the target protein by comparing the SRM signature peak area for a given target peptide to the SRM signature peak areas from other fragment peptides derived from different proteins within the same biological sample in order to normalize changing levels of histones protein to levels of other proteins that do not change their levels of expression under various cellular conditions.

4. These assays can be applied to both unmodified fragment peptides and to modified fragment peptides of the target proteins, where the modifications include, but are not limited to phosphorylation and/or glycosylation, acetylation, methylation (mono, di, tri), citrullination, ubiquitinylation and where the relative levels of modified peptides are determined in the same manner as determining relative amounts of unmodified peptides.

Absolute quantification of a given peptide may be achieved by:

1. Comparing the SRM/MRM signature peak area for a given fragment peptide from the target proteins in an individual biological sample to the SRM/MRM signature peak area of an internal fragment peptide standard spiked into the protein lysate from the biological sample. The internal standard may be a labeled synthetic version of the fragment peptide from the target protein that is being interrogated or the labeled recombinant protein. This standard is spiked into a sample in known amounts before (mandatory for the recombinant protein) or after digestion, and the SRM/MRM signature peak area can be determined for both the internal fragment peptide standard and the native fragment peptide in the biological sample separately, followed by comparison of both peak areas. This can be applied to unmodified fragment peptides and modified fragment peptides, where the modifications include but are not limited to phosphorylation and/or glycosylation, acetylation, methylation (e.g. mono-, di-, or tri-methylation), citrullination, ubiquitinylation, and where the absolute levels of modified peptides can be determined in the same manner as determining absolute levels of unmodified peptides.

2. Peptides can also be quantified using external calibration curves. The normal curve approach uses a constant amount of a heavy peptide as an internal standard and a varying amount of light synthetic peptide spiked into the sample. A representative matrix similar to that of the test samples needs to be used to construct standard curves to account for a matrix effect. Besides, reverse curve method circumvents the issue of endogenous analyte in the matrix, where a constant amount of light peptide is spiked on top of the endogenous analyte to create an internal standard and varying amounts of heavy peptide are spiked to create a set of concentration standards. Test samples to be compared with either the normal or reverse curves are spiked with the same amount of standard peptide as the internal standard spiked into the matrix used to create the calibration curve.

The invention further relates to kits, the use of the kits and methods wherein such kits are used. The invention relates to kits for carrying out the herein above and below provided methods. The herein provided definitions, e.g. provided in relation to the methods, also apply to the kits of the invention.

In particular, the invention relates to kits for diagnosis, prognosis, prediction, risk assessment and/or risk stratification of preterm birth (PTB) in a pregnant subject and kits for carrying out the method of the invention, comprising:
detection reagents for determining a level of MMP9 or fragment(s) thereof and/or PAPP-A2 or fragment(s) thereof in a sample from a pregnant subject,
and optionally, detection reagents for determining a level of at least one additional biomarker or fragment(s) thereof, preferably selected from the group comprising or consisting of LTF, CGA, INHA, ACTA, NOTUM, PAPP-A, PSG3, MST1L, SHBG, sFlt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1 and MMP8 in a sample from a patient, and
reference data, or means to obtain reference data, for the risk of whether PTB in a pregnant subject will occur, wherein the reference data comprise reference levels for MMP9 and/or PAPP-A2, and optionally additionally reference levels for said at least one additional parameter or biomarker,
wherein said reference data is preferably stored on a computer readable medium and/or employed in the form of a computer executable code, such as an algorithm, configured for comparing the determined levels of MMP9 or fragment(s) thereof and/or PAPP-A2 or fragment(s) thereof and optionally of the additional biomarker or fragment(s) thereof, with the provided reference levels.

As used herein, “reference data” comprise reference level(s) of MMP9 and/or PAPP-A2 and optionally further biomarkers of clinical scores as disclosed herein. The levels of respective biomarkers or parameters can be compared to the reference levels comprised in the reference data of the kit. The reference levels are herein described above and are exemplified also in the appended examples. The reference data can also include a reference sample to which the level of MMP9 and/or PAPP-A2 or other optional markers can be compared. The reference data can also include an instruction manual how to use the kits of the invention.

The kit may additionally comprise items useful for obtaining a sample, such as a blood sample, for example the kit may comprise a container, wherein said container comprises a device for attachment of said container to a cannula or syringe, is a syringe suitable for blood isolation, exhibits an internal pressure less than atmospheric pressure, such as is suitable for drawing a pre-determined volume of sample into said container, and/or comprises additionally detergents, chaotropic salts, ribonuclease inhibitors, chelating agents, such as guanidinium isothiocyanate, guanidinium hydrochloride, sodium dodecylsulfate, polyoxyethylene sorbitan monolaurate, RNAse inhibitor proteins, and mixtures thereof, and/or A filter system containing nitro-cellulose, silica matrix, ferromagnetic spheres, a cup retrieve spill over, trehalose, fructose, lactose, mannose, poly-ethylen-glycol, glycerol, EDTA, TRIS, limonene, xylene, benzoyl, phenol, mineral oil, anilin, pyrol, citrate, and mixtures thereof.

As used herein, the “detection reagent” or the like are reagents that are suitable to determine the herein described marker(s). Such exemplary detection reagents are, for example, ligands, e.g. antibodies or fragments thereof, which specifically bind to the peptide or epitopes of the herein described marker(s). Such ligands might be used in immunoassays as described above. Further reagents that are employed in the immunoassays to determine the level of the marker(s) may also be comprised in the kit and are herein considered as detection reagents. Detection reagents can also relate to reagents that are employed to detect the markers or fragments thereof by MS based methods. Such detection reagent can thus also be reagents, e.g. enzymes, chemicals, buffers, etc, that are used to prepare the sample for the MS analysis. A mass spectrometer can also be considered as a detection reagent. Detection reagents according to the invention can also be calibration solution(s), e.g. which can be employed to determine and compare the level of the marker(s).

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are often calculated by plotting the value of a variable versus its relative frequency in “normal” (i.e. apparently healthy individuals not having an increased risk of PTB and a risk population subjects that are known to have later developed PTB). For any particular marker, a distribution of marker levels for subjects with and without an increased risk of PTB will likely overlap. Under such conditions, a test does not absolutely distinguish normal from risk/disease with 100% accuracy, and the area of overlap might indicate where the test cannot distinguish normal from disease. A threshold can be selected, below which the test is considered to be abnormal and above which the test is considered to be normal or below or above which the test indicates a specific condition, e.g. increased risk of PTB. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art; see, e.g., Hanley et al. 1982. Radiology 143:29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

Thus, the terms “comprising”/“including”/“having” mean that any further component (or likewise features, integers, steps and the like) can/may be present. The term “consisting of” means that no further component (or likewise features, integers, steps and the like) is present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means those specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

### EXAMPLES

The invention is further described by the following examples. These are not intended to limit the scope of the invention, but represent preferred embodiments of aspects of the invention provided for greater illustration of the invention described herein.

### Materials and Methods of the Examples

### Study Population

The serum from nulliparous women with singleton pregnancy were longitudinally collected from 28 subjects who underwent normal pregnancies (NP) and 66 subjects who subsequently developed a spontaneous preterm birth (sPTB). sPTB is defined as spontaneous delivery before 37 weeks of gestation without any indication for induction of delivery. Early preterm birth is defined as spontaneous delivery before 34 weeks of gestation without any induction of delivery (sPTB<34 weeks). Very early preterm birth is defined as as spontaneous delivery before 32 weeks of gestation without any induction of delivery (sPTB<32 weeks). All pregnant women signed a consent form, approved by Laval University Ethics Committee.

Serum samples were collected during routine hospital visits at 1^{st} trimester of pregnancy between 11 and 13 weeks of gestation, at 2^{nd} trimester between 20 and 24 weeks of gestation and at 3^{rd} trimester between 30 to 34 weeks of gestation. Patient's characteristics associated to the serum samples have been collected including maternal age, gestational age at collection, body mass index (BMI, prior to pregnancy), ethnicity, birth weight and percentage of perinatal death.

### Biomarkers Measurements

The list of biomarkers evaluated during the study is presented in Table 2.

ELISA: PAPP-A2, and Activin A serum concentrations were determined using ELISA Kits (Cat. Number AL-109 and Cat. Number AL-110 respectively, Ansh Labs, Webster, TX, USA) according to manufacturer's instructions. Absorbance measurements were performed on PHERAstar microplate reader (BMG Labtechnologies, GmbH, Offenburg, Germany).

KRYPTOR: CGA, INHA, PAPP-A and sFlt-1 serum concentrations were measured on the fully automated B-R-A-H-M-S™ KRYPTOR™ instrument (Thermo Fisher Scientific, B-R-A-H-M-S GmbH, Hennigsdorf/Berlin, Germany) using an advanced homogeneous sandwich fluoroimmuno-assay commercialized under the references CgA II #839.050, Inhibin A #850.075, PAPP-A #866.075 and sFlt-1 #845.075.

The system uses the sensitive Time Resolved Amplified Cryptate Emission (TRACE™) technology based on a non-radiative energy transfer between a donor (Europium Cryptate or Terbium) and an acceptor.

Mass Spectrometry: The levels of MMP9, LTF, NOTUM, PSG3, MST1L, SHBG, ADA12 and FCN3 were determined in the serum samples by Selected Reaction Monitoring (SRM) assays. SRM assays is the targeted measurement of specific peptides derived from the biomarkers by LC-MS/MS technology (Ultimate 3000 UHPLC and TSQ Quantiva mass spectrometer (MS); ThermoFisher Scientific).

The sequence of peptides measured by SRM/Mass Spectrometry assays for each biomarker is presented in Table 3.

A first step of optimization is done to select peptide sequences and fragmentation ions thereof, so-called transitions, useful surrogates for monitoring marker proteins levels in the serum sample. This process was done on standard synthetic peptides, corresponding to the 20 candidates mentioned above, which are isotopically heavy labeled incorporating 13C- and 15N-labeled arginine or lysine (Pepscan).

All patient serum sample were digested with trypsin protease following digestion protocol described by Incamps et al., 2018. Standard heavy peptides were then added to each of digested samples before injection.

Samples were injected on HPLC system for peptides separation by reversed phase on an Accucore aQ column. Then separated peptides and associated transitions were monitored by SRM. The measurement was done following SRM developed method described by Incamps et al., 2018.

SRM assays were analyzed with Skyline (MacCoss Lab) software. Peptides were first identified by co-eluting light and heavy-labeled transitions on the chromatogram. Light and heavy chromatographic peaks corresponding to these transitions were then integrated in order to calculate an area under the curve for each transition. A sum of each transition's area is calculated for light and for heavy peptides. The concentration of biomarkers is expressed as relative abundance (RA) corresponding to the ratio of the endogenous peptide area divided by the heavy peptide area multiplied by 1000.

### Statistical Analysis

The statistical analysis was performed using JMP® statistical software version 13.0.0 (SAS Institute Inc., Cary, NC, USA) and Analyse-it® for Miscrosoft® Excel 4.65.3. Data are presented as the median±standard deviation. Differences between groups were analyzed using the Mann-Whitney U test (adjusted significance level P<0.05).

The predictive performance of biomarkers and combination of biomarkers was evaluated by logistic regression and ROC curve analysis. Biomarker concentrations were corrected for gestational age by adding this parameter in the logistic regression. The detection rate (DR) was calculated for a false positive rate fixed at 10%.

### Results of the Examples

The population was stratified according to the gestational age at collection and to the gestational age at delivery for sPTB patients (<37 weeks, <34 weeks and <32 weeks of gestation).

Patients' characteristics are summarized in Table 1. No significant difference in maternal age or gestational age between normal pregnancies and sPTB patients is observed (maternal age: 1^{st} trimester, sPTB<32 weeks p=0.9, sPTB<34 weeks p=0.9, all sPTB p=0.6; 2^{nd} trimester, sPTB<34 weeks p=0.9, all sPTB p=0.6; 3^{rd} trimester, sPTB<34 weeks p=0.9, all sPTB p=0.6; gestational age: 1^{st} trimester, sPTB<32 weeks p=0.9, sPTB<34 weeks p=0.9, all sPTB p=0.6; 2^{nd} trimester, sPTB<34 weeks p=0.9, all sPTB p=0.6; 3^{rd} trimester, sPTB<34 weeks p=0.9, all sPTB p=0.6).

Fourteen biomarkers have been measured in the study (Table 2) and eight of them were measured by SRM (Table 3).

During the 1^{st} trimester of pregnancy, the median concentration of MMP9 is significantly increased in all patients who will undergo a sPTB compared to patients with normal pregnancies (Table 4). This increase is highest in patients who will undergo an early preterm birth (sPTB<34 weeks) or a very early preterm birth (sPTB<32 weeks). The increase in MMP9 concentration is also observed during 2^{nd} trimester in all sPTB and early sPTB. A similar trend is observed during 3^{rd} trimester although it doesn't reach statistical significance.

On the contrary, PAPP-A2 median concentration is not increased in sPTB patients compared to normal pregnancies during the 1^{st} trimester. A significant increase is observed during 2nd trimester in all sPTB and early sPTB (sPTB<34 weeks) compared to normal pregnancies and reaches the highest statistical significance during 3^{rd} trimester (Table 4).

Higher concentration of MMP9 during first trimester of pregnancy allows the prediction of 44% of sPTB, 53% of sPTB<34 weeks and 78% of sPTB<32 weeks of gestation (Table 5). During second trimester of pregnancy, it allows the detection of 33% of sPTB and 71% of sPTB<34 weeks. During 3^{rd} trimester it allows the prediction of 48% of sPTB and 67% of sPTB<34 weeks.

Higher concentration of PAPP-A2 during first trimester of pregnancy allows the prediction of 20% of sPTB, 21% of sPTB<34 weeks and 28% of sPTB<32 weeks of gestation (Table 5). During second trimester of pregnancy, it allows the detection of 17% of sPTB and 29% of sPTB<34 weeks. During 3^{rd} trimester, the predictive value of PAPP-A2 is the highest as it allows the prediction of 61% of sPTB and 100% of sPTB<34 weeks.

Then MMP9 and PAPP-A2 are complementary biomarkers which allow the prediction of sPTB throughout pregnancy course.

The predictive value of MMP9 and PAPP-A2 is even increased when combined with each other or with a second biomarker as shown by AUC and DR (Table 6).

During 1^{st} trimester the DR of sPTB<32 weeks is increased by 11% reaching 89% when combining MMP9 to PAPP-A or NOTUM, the DR of sPTB<34 weeks is increased by 9% reaching 62% when combining MMP9 to PAPP-A2 and the DR of all sPTB is increased by 6% reaching 50% when combining PAPP-A2 to CGA.

During 2^{nd} trimester the DR of sPTB<34 weeks is increased by 15% reaching 86% when combining MMP9 to PAPP-A2, MST1L or PSG3 and the DR of all sPTB is increased by 21% reaching 54% when combining PAPP-A2 to PAPP-A.

During 3^{rd} trimester the DR of sPTB<34 weeks cannot be increased as it already reached 100%, however the AUC is improved from 0.976 to the maximum value of 1.000 when combining PAPP-A2 to PAPP-A or SHBG, which suggests an improvement of the detection. The DR of all sPTB is increased by 17% reaching 78% when combining PAPP-A2 to SHBG.

During 1^{st} trimester, in parcular week 11-13, the median of MMP9 levels is 1.5 fold increased for sPTB<32 weeks, 1.5 for increased for sPTB<34 weeks, and 1.3 fold increased for all sPTB, as compared to normal pregnancies.

During 2^{st} trimester, in parcular week 20-24, the median of MMP9 levels is 1.6 fold increased for SPTB<34 weeks, and 1.2 fold increased for all sPTB, as compared to normal pregnancies.

During 2^{st} trimester, in parcular week 20-24, the median of PAPP-A2 levels is 2 fold increased for sPTB<34 weeks, and 1.3 fold increased for all sPTB, as compared to normal pregnancies.

During 3^{st} trimester, in parcular week 30-34, the median of MMP9 levels is 1.4 fold increased for sPTB<34 weeks, and 1.2 fold increased for all sPTB, as compared to normal pregnancies.

During 3^{st} trimester, in parcular week 30-34, the median of PAPP-A2 levels is 3.6 fold increased for sPTB<34 weeks, and 2.2 fold increased for all sPTB, as compared to normal pregnancies.

### CONCLUSIONS

PAPP-A2 and MMP9 are predictive biomarkers for spontaneous preterm birth throughout pregnancy. They allow, as single biomarker or in combination, the identification of 89% of very early early preterm birth (<32 weeks) as soon as 1^{st} trimester, 86% of early birth (<34 weeks) during 2^{nd} trimester and 100% of early birth (<34 weeks) during 3^{rd} trimester. In addition they allow the detection of 50% to 78% of all preterm birth from 1^{st} through 3^{rd} trimester.

### Tables of the Examples

TABLE 1
Characteristics in the study population: Values are presented
as medians (interquartile range) or numbers (%).

| | Normal | sPTB <32 | sPTB <34 | All |
|---|---|---|---|---|
| | pregnancies | weeks | weeks | sPTB |
| Characteristics | 11-13 weeks' gestation | | | |
| n | 27 | 18 | 34 | 66 |
| Maternal age in | 28 | 29 | 29 | 29 |
| years, meadian (IQR) | (26-32) | (27-32) | (27-31) | (27-32) |
| Body mass index | 22.9 | 26.1 | 26.1 | 25.05 |
| (BMI), median (IQR) | (21.1-27.6) | (21.75-30.5) | (21.9-31.7) | (21.9-30.4) |
| Ethnicity | | | | |
| Caucasian, n (%) | 26 | 17 | 32 | 62 |
| | (96.3%) | (94.4%) | (94.1%) | (93.9%) |
| Other, n (%) | 1 | n = 1 | 2 | 4 |
| | (3.7%) | (5.6%) | (5.9%) | (6.1%) |
| Gestational age at | 12.71 | 13 | 13 | 12.86 |
| screening in weeks, | (12.29-13.14) | (12.43-13.43) | (12.43-13.33) | (12.43-13.29) |
| median (IQR) | | | | |
| Gestational age at | 40.14 | 28.64 | 31.79 | 33.86 |
| delivery in weeks, | (39.43-40.71) | (26.4-30.47)* | (28.43-33.57)* | (31.61-34.86)* |
| median (IQR) | | | | |
| Birth weight in | 3570 | 1310 | 1745 | 2270 |
| grams, median (IQR) | (3310-3710) | (925-1542.5)* | (1260-2267.5)* | (1697.5-2485)* |
| Perinatal death, n (%) | — | 1 | 4 | 4 |
| | | (5.6%) | (11.8%) | (6.1%) |
| | 20-24 weeks' gestation | | | |
| n | 28 | — | 8^{a} | 25^{a} |
| Maternal age in | 28 | — | 29.5 | 29 |
| years, meadian (IQR) | (26-31.5) | | (27-31) | (27-31.5) |
| Body mass index | 23.05 | — | 23.1 | 23.7 |
| (BMI), median (IQR) | (21.2-27.58) | | (21.25-25.23) | (22.5-29.35) |
| Ethnicity | | | | |
| Caucasian, n (%) | 26 | — | 8 | 24 |
| | (92.9%) | | (100%) | (96%) |
| Other, n (%) | 2 | — | — | 1 |
| | (7.1%) | | | (4%) |
| Gestational age at | 21.57 | — | 21.86 | 21.57 |
| screening in weeks, | (21.14-23.21) | | (21.14-23) | (21.14-23) |
| median (IQR) | | | | |
| Gestational age at | 40.14 | — | 33.29 | 35.14 |
| delivery in weeks, | (39.43-40.68) | | (31.43-33.71)* | (33.71-36.43)* |
| median (IQR) | | | | |
| Birth weight in | 3574 | — | 2055 | 2480 |
| grams, median (IQR) | (3337.5-3707.5) | | (1632.5-2365)* | (2240-2830)* |
| Perinatal death, n (%) | — | — | — | — |
| | 30-34 weeks' gestation | | | |
| n | 28 | — | 6 | 23 |
| Maternal age in | 28 | — | 30.5 | 29 |
| years, meadian (IQR) | (26-31.5) | | (26.5-31) | (27-32) |
| Body mass index | 23.05 | — | 23.1 | 23.7 |
| (BMI), median (IQR) | (21.2-27.58) | | (21.95-28) | (22.7-30.3) |
| Ethnicity | | | | |
| Caucasian, n (%) | 26 | — | 6 | 22 |
| | (92.9%) | | (100%) | (95.7%) |
| Other, n (%) | 2 | — | — | 1 |
| | (7.1%) | | | (4.3%) |
| Gestational age at | 32.07 | — | 31.86 | 31.86 |
| screening in weeks, | (31.36-32.57) | | (30.89-32.43) | (31-33) |
| median (IQR) | | | | |
| Gestational age at | 40.14 | — | 33.57 | 35.14 |
| delivery in weeks, | (39.43-40.68) | | (32.82-33.75)* | (33.86-36.43)* |
| median (IQR) | | | | |
| Birth weight in | 3574 | — | 2245 | 2500 |
| grams, median (IQR) | (3337.5-3707.5) | | (1697.5-2407.5)* | (2290-2900)* |
| Perinatal death, n (%) | — | — | — | — |

^{a}Gestational age missing for 1 sample;
*Statistically significant difference compared to normal pregancies (p-value < 0.05)

TABLE 2
Biomarkers list.

| | | Accession | | |
|---|---|---|---|---|
| | | number | | |
| Symbol | Protein names | UniProt | Gene | Entry name |
| MMP9 | Matrix | P14780 | MMP9 | MMP9_HUMAN |
| | metalloproteinase-9 | | | |
| LTF | Lactotransferrin | P02788 | LTF | TRFL_HUMAN |
| CGA | Chromogranin-A | P10645 | CHGA | CMGA_HUMAN |
| INHA^{a} | Inhibin alpha chain | P05111 | INHA | INHA_HUMAN |
| | Inhibin beta A chain | P08476 | INHBA | INHBA_HUMAN |
| PAPP-A2 | Pappalysin-2 | Q9BXP8 | PAPPA2 | PAPP2_HUMAN |
| ACTA | Activin A/ | P08476 | INHBA | INHBA_HUMAN |
| | homodimer of Inhibin | | | |
| | beta A chain | | | |
| NOTUM | Palmitoleoyl-protein | Q6P988 | NOTUM | NOTUM_HUMAN |
| | carboxylesterase | | | |
| | NOTUM | | | |
| PAPP-A | Pregnancy- | Q13219 | PAPPA | PAPP1_HUMAN |
| | associated plasma | | | |
| | protein A, | | | |
| | pappalysin 1 | | | |
| PSG3 | Pregnancy-specific | Q16557 | PSG3 | PSG3_HUMAN |
| | beta-1-glycoprotein 3 | | | |
| MST1L | Putative macrophage | Q2TV78 | MST1L | MST1L_HUMAN |
| | stimulating 1-like | | | |
| | protein | | | |
| SHBG | Sex hormone- | P04278 | SHGB | SHBG_HUMAN |
| | binding globulin | | | |
| sFlt-1 | Vascular endothelial | P17948 | FLT1 | VGFR1_HUMAN |
| | growth factor | | | |
| | receptor 1 | | | |
| ADA12 | Disintegrin and | O43184 | ADAM12 | ADA12_HUMAN |
| | metalloproteinase | | | |
| | domain-containing | | | |
| | protein 12 | | | |
| FCN3 | Ficolin-3 | O75636 | FCN3 | FCN3_HUMAN |

^{a}Inhibin A is a heterodimer of beta-A and alpha chain

TABLE 3
List of peptides for SRM/Mass Spectrometry assays.

| | | Accession | | | |
|---|---|---|---|---|---|
| | | number | | | Peptide |
| Symbol | Protein name | UniProt | Gene | Entry name | sequence |
| MMP9 | Matrix | P14780 | MMP9 | MMP9_HUMAN | LGLGADVAQVTGALR |
| | metalloproteinase-9 | | | | (SEQ ID NO 1) |
| LTF | Lactotransferrin | P02788 | LTF | TRFL_HUMAN | CGLVPVLAENYK |
| | | | | | (SEQ ID NO 2) |
| NOTUM | Palmitoleoyl-protein | Q6P988 | NOTUM | NOTUM_HUMAN | LGYPAIQVR |
| | carboxylesterase | | | | (SEQ ID NO 3) |
| | NOTUM | | | | |
| PSG3 | Pregnancy-specific | Q16557 | PSG3 | PSG3_HUMAN | VSAPSGTGHLPGLNPL |
| | beta-1-glycoprotein 3 | | | | (SEQ ID NO 4) |
| MST1L | Putative macrophage | Q2TV78 | MST1L | MST1L_HUMAN | CEIAGWGETK |
| | stimulating | | | | (SEQ ID NO 5) |
| | 1-like protein | | | | |
| SHBG | Sex hormone-binding | P04278 | SHGB | SHBG_HUMAN | LDVDQALNR |
| | globulin | | | | (SEQ ID NO 6) |
| ADA12 | Disintegrin and | O43184 | ADA12 | ADA12_HUMAN | NHPEVLNIR |
| | metalloproteinase | | | | (SEQ ID NO 7) |
| | domain-containing | | | | |
| | protein 12 | | | | |
| FCN3 | Ficolin-3 | O75636 | FCN3 | FCN3_HUMAN | YAVSEAAAHK |
| | | | | | (SEQ ID NO 8) |

TABLE 4
Biomarker concentrations differences between patient groups.

| | Normal | | | | | | | | | |
|---|---|---|---|---|---|---|---|---|---|---|
| | pregnancies | sPTB <32 weeks | | | sPTB <34 weeks | | | All sPTB | | |
| | 11-13 weeks' gestation | | | | | | | | | |
| | 27 | 18 | | | 34 | | | 66 | | |
| | Mean ± | Median ± | p- | | Median ± | p- | | Median ± | p- | |
| n | SD | SD | value | FC | SD | value | FC | SD | value | FC |
| MMP9 (Relative | 13.4 ± | 20.5 ± | p < | 1.5 | 19.5 ± | p < | 1.5 | 17.2 ± | p < | 1.3 |
| Abundance) | 6.2 | 8.3 | 0.001 | | 8.0 | 0.01 | | 8.1 | 0.05 | |
| PAPP-A2 | 14.1 ± | 13.8 ± | p = | 1.0 | 13.5 ± | p = | 1.0 | 14.1 ± | p = | 1.0 |
| (ng/mL) | 7.0 | 8.4 | 0.6 | | 7.6 | 0.8 | | 7.8 | 0.4 | |
| | 20-24 weeks' gestation | | | | | | | | | |
| | 28 | — | | | 8 | | | 25 | | |
| | Median ± | Median ± | p- | | Median ± | p- | | Median ± | p- | |
| n | SD | SD | value | FC | SD | value | FC | SD | value | FC |
| MMP9 (Relative | 16.0 ± | — | — | — | 26.1 ± | p < | 1.6 | 19.9 ± | p < | 1.2 |
| Abundance) | 6.1 | | | | 10.9 | 0.01 | | 9.72 | 0.05 | |
| PAPP-A2 | 17.5 ± | — | — | — | 35.5 ± | p < | 2.0 | 23.0 ± | p < | 1.3 |
| (ng/mL) | 14.7 | | | | 16.6 | 0.05 | | 15.4 | 0.05 | |
| | 30-34 weeks' gestation | | | | | | | | | |
| | 28 | — | | | 6 | | | 23 | | |
| | Median ± | Median ± | p- | | Median ± | p- | | Median ± | p- | |
| n | SD | SD | value | FC | SD | value | FC | SD | value | FC |
| MMP9 (Relative | 15.7 ± | — | — | — | 21.8 ± | p = | 1.4 | 19.3 ± | p = | 1.2 |
| Abundance) | 5.8 | | | | 9.0 | 0.2 | | 10.2 | 0.2 | |
| PAPP-A2 | 44.9 ± | — | — | — | 160.8 ± | p < | 3.6 | 99.4 ± | p < | 2.2 |
| (ng/mL) | 28.8 | | | | 88.3 | 0.001 | | 88.1 | 0.005 | |

TABLE 5
Predictive performance of single biomarkers
corrected for gestational age.

| | sPTB <32 weeks | | sPTB <34 weeks | | All sPTB | |
|---|---|---|---|---|---|---|
| | 11-13 weeks' gestation | | | | | |
| | 18 sPTB vs 27 NP | | 34 sPTB vs 27 NP | | 66 sPTB vs 27 NP | |
| | | DR | | DR | | DR |
| | AUC | (%) | AUC | (%) | AUC | (%) |
| MMP9 | 0.887 | 78 | 0.814 | 53 | 0.710 | 44 |
| PAPP- | 0.648 | 28 | 0.608 | 21 | 0.586 | 20 |
| A2 | | | | | | |
| | sPTB <32 weeks | | sPTB <34 weeks | | All sPTB | |
| | 20-24 weeks' gestation | | | | | |
| | — | | 7 sPTB vs 28 NP | | 24 sPTB vs 28 NP | |
| | | DR | | DR | | DR |
| | AUC | (%) | AUC | (%) | AUC | (%) |
| MMP9 | — | — | 0.816 | 71 | 0.655 | 33 |
| PAPP- | — | — | 0.745 | 29 | 0.644 | 17 |
| A2 | | | | | | |
| | sPTB <32 weeks | | sPTB <34 weeks | | All sPTB | |
| | 30-34 weeks' gestation | | | | | |
| | — | | 6 sPTB vs 28 NP | | 23 sPTB vs 28 NP | |
| | | DR | | DR | | DR |
| | AUC | (%) | AUC | (%) | AUC | (%) |
| MMP9 | — | — | 0.714 | 67 | 0.610 | 48 |
| PAPP- | — | — | 0.976 | 100 | 0.775 | 61 |
| A2 | | | | | | |

TABLE 6
Predictive performance of combination of biomarkers corrected for
gestational age (Bold: improvement compared to single biomarker).

| | sPTB <32 weeks | | sPTB <34 weeks | | All sPTB | |
|---|---|---|---|---|---|---|
| | 11-13 weeks' gestation | | | | | |
| | 18 sPTB vs 27 NP | | 34 sPTB vs 27 NP | | 66 sPTB vs 27 NP | |
| | AUC | DR (%) | AUC | DR (%) | AUC | DR (%) |
| MMP9 + NOTUM | 0.916 | 89 | 0.812 | 50 | 0.701 | 42 |
| MMP9 + PAPP-A | 0.909 | 89 | 0.812 | 53 | 0.704 | 45 |
| MMP9 + PAPP-A2 | 0.885 | 83 | 0.827 | 62 | 0.718 | 47 |
| PAPP-A2 + LTF | 0.823 | 67 | 0.790 | 59 | 0.702 | 48 |
| PAPP-A2 + CGA | 0.716 | 61 | 0.670 | 50 | 0.653 | 50 |
| | sPTB <32 weeks | | sPTB <34 weeks | | All sPTB | |
| | 20-24 weeks' gestation | | | | | |
| | — | | 7 sPTB vs 28 NP | | 24 sPTB vs 28 NP | |
| | AUC | DR (%) | AUC | DR (%) | AUC | DR (%) |
| MMP9 + MST1L | — | — | 0.857 | 86 | 0.699 | 42 |
| MMP9 + PAPP-A2 | — | — | 0.857 | 86 | 0.692 | 46 |
| MMP9 + PSG3 | — | — | 0.847 | 86 | 0.656 | 50 |
| PAPP-A2 + INHA | — | — | 0.827 | 71 | 0.716 | 46 |
| PAPP-A2 + CGA | | | 0.816 | 43 | 0.688 | 46 |
| PAPP-A2 + PAPP-A | | | 0.781 | 57 | 0.682 | 54 |
| | sPTB <32 weeks | | sPTB <34 weeks | | All sPTB | |
| | 30-34 weeks' gestation | | | | | |
| | — | | 6 sPTB vs 28 NP | | 23 sPTB vs 28 NP | |
| | AUC | DR (%) | AUC | DR (%) | AUC | DR (%) |
| MMP9 + PAPP-A2 | — | — | 0.976 | 100 | 0.792 | 57 |
| MMP9 + ACTA | — | — | 0.899 | 83 | 0.693 | 43 |
| MMP9 + ADA12 | — | — | 0.851 | 83 | 0.724 | 52 |
| MMP9 + FCN3 | — | — | 0.804 | 83 | 0.758 | 52 |
| PAPP-A2 + SHBG | — | — | 1.000 | 100 | 0.843 | 78 |
| PAPP-A2 + PAPP-A | — | — | 1.000 | 100 | 0.798 | 74 |
| PAPP-A2 + sFlt-1 | — | — | 0.988 | 100 | 0.787 | 70 |
| PAPP-A2 + MST1L | — | — | 0.976 | 100 | 0.877 | 74 |

### REFERENCES

Beta, J., R. Akolekar, W. Ventura, A. Syngelaki and K. H. Nicolaides (2011). “Prediction of spontaneous preterm delivery from maternal factors, obstetric history and placental perfusion and function at 11-13 weeks.” Prenat Diagn 31 (1): 75-83.
Blencowe, H., S. Cousens, D. Chou, M. Oestergaard, L. Say, A. B. Moller, M. Kinney, J. Lawn and G. Born Too Soon Preterm Birth Action (2013). “Born too soon: the global epidemiology of 15 million preterm births.” Reprod Health 10 Suppl 1: S2.
Chawanpaiboon, S., J. P. Vogel, A. B. Moller, P. Lumbiganon, M. Petzold, D. Hogan, S. Landoulsi, N. Jampathong, K. Kongwattanakul, M. Laopaiboon, C. Lewis, S. Rattanakanokchai, D. N. Teng, J. Thinkhamrop, K. Watananirun, J. Zhang, W. Zhou and A. M. Gulmezoglu (2019). “Global, regional, and national estimates of levels of preterm birth in 2014: a systematic review and modelling analysis.” Lancet Glob Health 7 (1): e37-e46.
Goldenberg, R. L., J. F. Culhane, J. D. Iams and R. Romero (2008). “Epidemiology and causes of preterm birth.” Lancet 371 (9606): 75-84.
Incamps, A., C. Saez-Boiteau, S. L. Tiede, P. Rebillard, J. Schulte, J. Vialaret, A. Beinrucker, S. Lehmann and C. Hirtz (2018). “Impact of biological matrix on inflammatory protein biomarker quantification based on targeted mass spectrometry.” Bioanalysis 10 (17): 1383-1399.
Korvenranta, E., L. Lehtonen, L. Rautava, U. Hakkinen, S. Andersson, M. Gissler, M. Hallman, J. Leipala, M. Peltola, O. Tammela, M. Linna and P. P. I. S. Group (2010). “Impact of very preterm birth on health care costs at five years of age.” Pediatrics 125 (5): e1109-1114.
Parry, S., H. Simhan, M. Elovitz and J. Iams (2012). “Universal maternal cervical length screening during the second trimester: pros and cons of a strategy to identify women at risk of spontaneous preterm delivery.” Am J Obstet Gynecol 207 (2): 101-106.
Quinn, J. A., F. M. Munoz, B. Gonik, L. Frau, C. Cutland, T. Mallett-Moore, A. Kissou, F. Wittke, M. Das, T. Nunes, S. Pye, W. Watson, A. A. Ramos, J. F. Cordero, W. T. Huang, S. Kochhar, J. Buttery and G. Brighton Collaboration Preterm Birth Working (2016). “Preterm birth: Case definition & guidelines for data collection, analysis, and presentation of immunisation safety data.” Vaccine 34 (49): 6047-6056.
Son, M. and E. S. Miller (2017). “Predicting preterm birth: Cervical length and fetal fibronectin.” Semin Perinatol 41 (8): 445-451.
Lee, S. M. et al. (2015), “Mid-trimester amniotic fluid pro-inflammatory biomarkers predict the risk of spontaneous preterm delivery in twins: a retrospective cohort study”, Journal of Perinatology 35 (8): 524-546
Parry, S. et al. (2019), “Markers of placental dysfunction and spontaneous preterm birth”, Amrocan Journal of obestrics & gynecology 222 (1)
Rasanen, J. et al., (2010), “Comprehensive Maternal Serum Proteomic Profiles of Preclinical and Clinical Preeclampsia”, 9 (8): 4274-4281
Shah, D. A. et al. (2015), “Bioactive factors in uteroplacental and systemic circulation link placental ischemia to generalized vascular dysfunction in hypertensive pregnancy and preeclampsia”, Biochemical Pharmacology, 95 (4): 211-226

1. Method for treating a subject to reduce the risk of preterm birth (PTB) in a pregnant subject, the method comprising:
determining a level of one or more biomarkers in a sample that has been isolated from said pregnant subject,
wherein the one or more biomarkers comprise at least one of matrix metallopeptidase 9 (MMP9) or fragment(s) thereof and Pappalysin-2 (PAPP-A2) or fragment(s) thereof,
wherein the level of the one or more biomarkers in the sample is indicative of the presence or absence of a subsequent PTB, and
treating said subject to decrease the risk of PTB.

2. Method according to claim 1, wherein the level of the one or more biomarkers in said sample is compared to a reference level of said biomarker.

3. Method according to claim 2, wherein an increased level of the one or more biomarkers in the pregnant subject as compared to the reference level is indicative of a subsequent PTB.

4. Method according to claim 1, wherein the PTB is spontaneous PTB (sPTB) or indicated preterm birth (iPTB).

5. Method according to claim 1, wherein PTB is early sPTB before gestational week 34 or very early sPTB before gestational week 32.

6. Method according to claim 1, wherein the sample has been isolated from the pregnant subject in the first, second or third trimester of pregnancy.

7. Method according to claim 1, wherein the pregnant subject is a nulliparous woman.

8. Method according to claim 1, wherein the pregnant subject shows no signs of PTB (asymptomatic subject).

9. Method according to claim 1, wherein the subject has a singleton pregnancy.

10. Method according to claim 1, wherein the sample is a bodily fluid sample selected from the group consisting of a blood sample, such as a venous blood sample, a capillary blood sample, a serum sample or a plasma sample, a vaginal fluid sample, a saliva sample and an amniotic fluid sample.

11. Method according to claim 1, wherein the method comprises determining a level of at least two biomarkers in a sample, wherein the at least two biomarkers comprise at least one of MMP9 and Pappalysin-2 (PAPP-A2) or fragment(s) thereof, and optionally one or more of LTF, CGA, INHA, ACTA, NOTUM, PAPP-A, PSG3, MST1L, SHBG, sFlt-1, ADA12 and FCN3, PIGF, PCT, MR-proADM, MIP-1a, MIP-1b, estriol, TNF-a, IL-6, IL-8, IL-1b, AFP, fFN, PAMG1, phIGFBP1, MMP8, or fragment(s) thereof.

12. Method according to claim 1, wherein the one or more biomarkers comprise MMP9 or fragment(s).

13. Method according to claim 12, comprising determining the levels MMP9 or fragment(s) thereof in a sample that has been isolated in the first or second trimester of pregnancy.

14. Method according to claim 12, comprising determining the levels MMP9 or fragment(s) thereof and at least one of NOTUM, PAPP-A, PAPP-A2, MST1L, PSG3, ACTA, ADA12 and FCN3 or fragment(s) thereof.

15. Method according to claim 1, wherein the one or more biomarkers comprise PAPP-A2 or fragment(s).

16. Method according to claim 15, comprising determining the levels PAPP-A2 or fragment(s) thereof in a sample that has been isolated in the second.

17. Method according to claim 15, comprising determining the levels PAPP-A2 or fragment(s) thereof and at least one of MMP9, LFT, CGA, INHA, PAPP-A, SHBG, sFlt-1 and MST1L or fragment(s) thereof.

18. Method according to claim 1, wherein the method additionally comprises treating a subject with an increased risk of PTB.

19. Method according to claim 1, wherein a method according to any one of the preceding claims is performed at least two times using samples that have been isolated from the pregnant subject over the first, second and/or third trimester of pregnancy.

20. Kit for carrying out the method of claim 1, comprising:
detection reagents for determining a level of MMP9 or fragment(s) thereof and/or PAPP-A2 or fragment(s) thereof in a sample from a pregnant subject,
and optionally, detection reagents for determining a level of at least one additional biomarker or fragment(s) thereof in a sample from a patient, and
reference data, or means to obtain reference data, for the risk of whether PTB in a pregnant subject will occur, wherein the reference data comprise reference levels for MMP9 and/or PAPP-A2, and optionally additionally reference levels for said at least one additional parameter or biomarker.

21. The method according to claim 2, wherein the reference level is derived from pregnant subjects without a prenatal disorder or condition.

22. The method according to claim 1, wherein the sample has been isolated from the pregnant subject in gestational weeks 9-13, 20-24, or 30-34.

23. Method according to claim 1 wherein the sample is a blood, serum or plasma sample.

24. The kit according to claim 20, wherein the at least one additional biomarker or fragment(s) thereof is selected from the group consisting of LTF, CGA, INHA, ACTA, NOTUM, PAPP-A, PSG3, MST1L, SHBG, sFlt-1, ADA12, FCN3, PIGF, PCT, MR-proADM, MIP-1a, MIP-1b, estriol, TNF-a, IL-6, IL-8, IL-1b, AFP, fFN, PAMG1, phIGFBP1 and MMP8.

25. The kit according to claim 20, wherein said reference data is stored on a computer readable medium and/or employed in the form of a computer executable code, such as an algorithm, configured for comparing the determined levels of MMP9 or fragment(s) thereof and/or PAPP-A2 or fragment(s) thereof and optionally of the additional biomarker or fragment(s) thereof, with the provided reference levels.

26. A method for determining a level of matrix metallopeptidase 9 (MMP9) or fragment(s) thereof and/or Pappalysin-2 (PAPP-A2) or fragment(s) thereof in a sample that has been isolated from a pregnant subject suspected to be at risk of preterm birth (PTB), comprising:
a. determining a level of matrix metallopeptidase 9 (MMP9) or fragment(s) thereof and/or Pappalysin-2 (PAPP-A2) or fragment(s) thereof in a sample that has been isolated from said pregnant subject,
b, wherein the sample is isolated from a subject in the first, second or third trimester of pregnancy, and
c. treating said subject to decrease the risk of PTB.

27. A complex comprising a binder against matrix metallopeptidase 9 (MMP9) or fragment(s) thereof and/or Pappalysin-2 (PAPP-A2) or fragment(s) thereof, bound to matrix metallopeptidase 9 (MMP9) or fragment(s) thereof or Pappalysin-2 (PAPP-A2) or fragment(s) thereof respectively, in a sample that has been isolated from a pregnant subject suspected to be at risk of preterm birth (PTB), wherein the sample is isolated from a subject in the first, second or third trimester of pregnancy.


SEQUENCE LISTING (8 sequences)

SEQ ID NO: 1 - protein, 15 aa, Homo sapiens
LGLGADVAQVTGALR

SEQ ID NO: 2 - protein, 12 aa, Homo sapiens
CGLVPVLAENYK

SEQ ID NO: 3 - protein, 9 aa, Homo sapiens
LGYPAIQVR

SEQ ID NO: 4 - protein, 16 aa, Homo sapiens
VSAPSGTGHLPGLNPL

SEQ ID NO: 5 - protein, 10 aa, Homo sapiens
CEIAGWGETK

SEQ ID NO: 6 - protein, 9 aa, Homo sapiens
LDVDQALNR

SEQ ID NO: 7 - protein, 9 aa, Homo sapiens
NHPEVLNIR

SEQ ID NO: 8 - protein, 10 aa, Homo sapiens
YAVSEAAAHK